(19) United States
(12) Patent Application Publication
(10) Pub. No.: US 2023/0129902 A1
(43) Pub. Date: Apr. 27, 2023

(54) Disease Prediction Using Analyte Measurement Features and Machine Learning

(71) Applicant: Dexcom, Inc., San Diego, CA (US)

(72) Inventors: Jee Hye Park, San Diego, CA (US); Spencer Troy Frank, San Diego, CA (US); David A. Price, San Diego, CA (US); Kazanna C. Hames, San Diego, CA (US); Charles R. Stroyeck, San Diego, CA (US); Joseph J. Baker, San Diego, CA (US); Arunachalam Panch Santhanam, San Diego, CA (US); Peter C. Simpson, San Diego, CA (US); Abdulrahman Jbaily, San Diego, CA (US); Justin Yi-Kai Lee, San Diego, CA (US); Qi An, San Diego, CA (US)

(73) Assignee: Dexcom, Inc., San Diego, CA (US)

(21) Appl. No.: 17/971,238

(22) Filed: Oct. 21, 2022

Related U.S. Application Data
(60) Provisional application No. 63/263,106, filed on Oct. 27, 2021.

(51) Int. Cl. G16H 50/30; G06N 20/00

(52) U.S. Cl. CPC G16H 50/30; G06N 20/00

(57) ABSTRACT

Disease prediction using analyte measurements and machine learning is described. In one or more implementations, a combination of features of analyte measurements may be selected from a plurality of features of the analyte measurements based on a robustness metric and a performance metric of the combination, and a machine learning model may be trained to predict a health condition classification using the combination. The performance metric may be associated with an accuracy of predicting the health condition classification, and the robustness metric may be associated with an insensitivity to analyte sensor manufacturing variabilities on the accuracy. Once trained, the machine learning model predicts the health condition classification for a user based on analyte measurements of the user collected by a wearable analyte monitoring device. The combination of features may be extracted from the analyte measurements of the user and input into the machine learning model to predict the classification.

### RELATED APPLICATION

This application claims the benefit of U.S. Provisional Patent Application No. 63/263,106, filed Oct. 27, 2021, and titled “Generalized Diagnostic Continuous Glucose Monitor,” the entire disclosure of which is hereby incorporated by reference.

### BACKGROUND

Some health conditions (e.g., medical conditions) produce or alter a level of analytes in blood and/or interstitial fluid. As an example, diabetes is a metabolic condition affecting hundreds of millions of people that results in elevated blood glucose levels. Although diabetes is one of the leading causes of death worldwide, with early detection and proper treatment, damage to the heart, blood vessels, eyes, kidneys, and nerves due to diabetes can be largely avoided.

Conventional tests for diabetes that are accepted by the clinical and regulatory communities include Hemoglobin A1c (HbA1c), Fasting Plasma Glucose (FPG), and 2-Hour Plasma Glucose (2Hr-PG). Both the FPG and the 2Hr-PG are part of the Oral Glucose Tolerance Test (OGTT), but the FPG can be tested separately from the OGTT. For the FPG test, a blood sample is taken, and the result is used to classify the person as being “normal” (e.g., no diabetes), as having prediabetes, or as having diabetes. Generally, a person is considered normal if her fasting glucose level is less than 100 milligrams per deciliter (mg/dL), whereas the person is classified as having prediabetes if her fasting blood glucose level is between 100 to 125 mg/dL or as having diabetes if her fasting blood glucose level greater than 126 mg/dL on two separate tests.

After measuring the person's fasting blood glucose for the FPG test, the OGTT then requires the person to drink a sugary liquid to cause a spike in the person's blood glucose level. Many people have difficulty tolerating this sugary drink, particularly women who are pregnant. The person's blood glucose levels are then tested periodically using additional blood samples for the next two hours for the 2Hr-PG. A blood sugar level that is less than 140 mg/dL is considered “normal”, whereas a reading of more than 200 mg/dL two hours after drinking the sugary drink indicates diabetes. A reading between 140 and 199 mg/dL indicates prediabetes.

Unlike the FPG and 2Hr-PG tests of the OGTT, which each measure a person's blood glucose level at a single point in time, the HbA1c test measures an average glucose level of the user over the previous two to three months. Rather than directly measuring glucose, however, the HbA1c test measures the percentage of glucose that is attached to hemoglobin. When glucose builds up in a person's blood, it attaches to hemoglobin, the oxygen-carrying protein in red blood cells. Red blood cells live for approximately two to three months in a person, and thus, the HbA1c test shows the average level of glucose in the blood over the previous two to three months. Like the FPG and 2Hr-PG tests, a blood sample is taken from the person and used to measure a person's HbA1c level. However, unlike the FPG and 2Hr-PG tests, the person does not need to be in a fasted state when the HbA1c test is administered. An HbA1c level of 6.5 percent or higher on two separate tests indicates that the person has diabetes, while an HbA1c level between 5.7 and 6.4 percent generally indicates that the person has prediabetes. An HbA1c level below 5.7 percent is considered normal.

Each of these conventional tests administered to screen for (or diagnose) diabetes has a variety of drawbacks that often lead to improper diagnosis. Conventional diabetes tests are often inaccurate because a given test administered to an individual on different days may result in inconsistent diagnoses due to various external factors causing glucose levels to fluctuate, such as sickness, stress, increased exercise, or pregnancy. In contrast, even though the HbA1c test measures an average glucose level over a previous two to three months, the HbA1c test results are greatly impacted by the user's blood glucose levels in the weeks leading up the test. As such, HbA1c test results can be greatly affected by changes in blood properties during the three-month time period, such as due to pregnancy or illness. Additionally, because the HbA1c test is not a direct measure of blood glucose, such tests may be inaccurate for people with various blood conditions such as anemia or those who have an uncommon form of hemoglobin.

Additionally, such conventional tests often have poor concordance. In other words, these tests do not necessarily detect diabetes in the same individuals. This lack of consistency between test types may lead to an inaccurate diagnosis or a failure to determine a proper treatment plan. For example, a user may have a high fasting glucose but an HbA1c score within the normal range. In such scenarios, different doctors may reach different conclusions regarding whether the user has diabetes as well as the type of treatment plan for the user.

Finally, there are also a variety of limitations and drawbacks of administering these tests to different people, such as pregnant women. For example, these conventional diabetes tests require the user to visit a doctor's office or lab in order to take a blood sample, which can be time consuming, expensive, and painful for some users. Each of these factors alone or in combination may act to create a psychological barrier preventing users from getting tested for diabetes, thereby mitigating the benefits associated with early detection. Moreover, many of these conventional tests require the user to be in a fasted state, which can be difficult, or even dangerous, for some users, including women who are pregnant.

### SUMMARY

To overcome these problems, health condition prediction, including diabetes prediction, using robustly accurate features extracted from analyte measurements and machine learning is leveraged. In one or more implementations, a combination of features of analyte measurements may be selected from a plurality of features of the analyte measurements based on a robustness metric and a performance metric of the combination, and a machine learning model may be trained to predict a health condition classification using the combination. The performance metric may be associated with an accuracy of predicting the health condition classification, and the robustness metric may be associated with an insensitivity to analyte sensor manufacturing variabilities on the accuracy. Once trained, the machine learning model predicts the health condition classification for a user based on analyte measurements of the user collected by a wearable analyte monitoring device. The combination of features may be extracted from the analyte measurements of the user and input into the machine learning model to predict the classification.

This Summary introduces a selection of concepts in a simplified form that are further described below in the Detailed Description. As such, this Summary is not intended to identify essential features of the claimed subject matter, nor is it intended to be used as an aid in determining the scope of the claimed subject matter.

### BRIEF DESCRIPTION OF THE DRAWINGS

The detailed description is described with reference to the accompanying figures.

FIG. 1 is an illustration of an environment in an example of an implementation that is operable to employ techniques described herein.

FIG. 2 depicts an example of a wearable analyte monitoring device in greater detail.

FIG. 3 depicts an example of an implementation in which analyte data is routed to different systems in connection with health condition classification.

FIG. 4 depicts an example of an implementation of a prediction system in greater detail in which a combination of extracted analyte features that is robust to analyte sensor variability and accurate for predicting a health condition classification is selected.

FIG. 5 shows example graphs illustrating analyte feature categorization and selection based on performance and robustness metrics.

FIG. 6 depicts an example of an implementation of a prediction system of in greater detail in which a machine learning model is trained to predict health condition classifications using a selected combination of extracted analyte features.

FIG. 7 depicts an example of an implementation of a prediction system of in greater detail in which a health condition classification is predicted using machine learning and a selected combination of extracted analyte features.

FIG. 8 depicts an example of an implementation of a user interface displayed for notifying a user about a health condition prediction that is generated based on analyte measurements collected during an observation period.

FIG. 9 depicts an example of an implementation of a user interface displayed for reporting a health condition prediction of a user along with other information produced in connection with the health condition prediction.

FIG. 10 depicts an example of an implementation of a user interface displayed for collecting additional data that can be used as input to machine learning models for generating a health condition prediction.

FIG. 11 depicts a procedure in an example of an implementation in which analyte features for robustly predicting a health condition classification are learned based on historical analyte measurements and outcome data of a user population.

FIG. 12 depicts a procedure in an example of an implementation in which a machine learning model is trained to predict a health condition classification based on historical analyte measurements and outcome data of a user population.

FIG. 13 depicts a procedure in an example of an implementation in which a machine learning model predicts a health condition classification based on analyte measurements of a user collected by a wearable analyte monitoring device during an observation period.

FIG. 14 shows an example plot of a percent time above a pre-determined glucose level and an interquartile range for participants having different diabetes classifications.

FIG. 15 illustrates an example of a system including various components of an example of a device that can be implemented as any type of computing device as described and/or utilized with reference to FIGS. 1-13 to implement embodiments of the techniques described herein.

### DETAILED DESCRIPTION

Overview

As mentioned above, conventional tests for diabetes that are accepted by the clinical and regulatory communities include Hemoglobin A1c (HbA1c), Fasting Plasma Glucose (FPG), and 2-Hour Plasma Glucose (2Hr-PG). Both the FPG and the 2Hr-PG can be part of the Oral Glucose Tolerance Test (OGTT), or the FPG can be tested separately from the OGTT. However, such conventional tests often have poor concordance with respect to detecting diabetes in the same individuals or may produce different results on different days due to the various factors that affect blood glucose levels. There are also a variety of limitations and drawbacks of administering these tests to different people, such as pregnant women.

Accordingly, machine learning may be leveraged for health condition prediction, such as diabetes prediction. The health condition prediction may be performed by processing analyte measurements (e.g., glucose measurements) obtained by an analyte monitoring device via one or more machine learning models (e.g., regression models, neural networks, reinforcement learning agents). For example, the analyte monitoring device may include a needle that is configured to be inserted through the skin of a user to contact the blood and/or interstitial fluid to measure, via a sensor of the monitoring device, one or more analytes relevant to identifying the health condition over an observation period. The one or more machine learning models are generated using historical analyte measurements and historical outcome data of a user population to predict a health condition classification (e.g., a diabetes classification) for an individual user. The historical analyte measurements of the user population may be provided by analyte monitoring devices worn by users of the user population. Furthermore, the historical outcome data used for training may vary depending on classifications that the machine learning models are configured to output. Generally, the historical outcome data includes one or more diagnostic measures obtained from sources independent of the analyte monitoring devices. For instance, the historical outcome data may indicate whether a respective user of the user population is clinically diagnosed with diabetes or not based on one or more diagnostic measures, such as HbA1c, FPG, or 2Hr-PG (or OGTT as a combination of FPG and 2Hr-PG). As such, the historical outcome data may indicate a clinically determined health condition classification.

Unlike conventional tests, which are traditionally administered in a lab or doctor's office, use of the wearable analyte monitoring device enables the analyte measurements to be collected remotely. For example, the analyte monitoring device may be mailed or otherwise provided to the user, e.g., from the provider of the analyte monitoring device, a pharmacy, a medical testing laboratory, a telemedicine service, and so forth. The user may then wear the analyte monitoring device over the course of the observation period, such as by continuously wearing the device at home and/or work.

The user can insert the sensor of the wearable analyte monitoring device into the user's body, such as by using an automatic sensor applicator. Unlike the blood draws required by conventional tests such as HbA1c, FPG, and 2Hr-PG, the user-initiated application of the analyte monitoring device is nearly painless and does not utilize a blood draw, consumption of a sugary drink, or fasting. Moreover, the automatic sensor applicator can enable the user to embed the sensor into the user's skin without the assistance of a clinician or health care provider. Although an automatic sensor applicator is discussed, the analyte monitoring device may be applied to or otherwise worn by the person in other ways without departing from the spirit or scope of the techniques described herein, such as without the automatic sensor applicator, with assistance of a health care professional (or a health care professional may simply apply the wearable to the person), or by peeling off a protective layer of an adhesive and affixing the adhesive to the person, to name just a few. Once the sensor is inserted into the user's skin, the analyte monitoring device monitors analyte levels of the person over the observation period, which may span multiple days. It is also to be appreciated that in some implementations, the sensor may not be inserted into the person's skin. Instead, the sensor may simply be disposed against the person's skin in such implementations, like an adhesive patch. Regardless, the sensor of the analyte monitoring device may continuously detect analytes and enable generation of analyte measurements.

However, the sensors of the analyte monitoring devices may have manufacturing variabilities that cause sensor-to-sensor differences in their responses to the analyte. For example, manufacturing variabilities may exist between different manufacturing lots, different sensor brands, different sensor models of a same brand, and the like. These manufacturing variabilities may result in different sensors producing different analyte measurements for a same analyte level in the blood or interstitial fluid, such as due to sensor bias (e.g., a positive or negative baseline offset). As a result, the one or more machine learning models may incorrectly predict a health condition state of some users if the models do not take manufacturing variabilities into account.

To overcome these problems, features representing, for example, values and patterns in the analyte measurements are extracted from the analyte measurements, and a robust combination of features that is insensitive to the manufacturing variabilities of the sensor is selected and used for predicting the health condition via the machine learning model. In one or more implementations, the robust combination of features is selected based on a robustness metric and a performance metric of each of a plurality of candidate combinations of features. The performance metric of each candidate combination of features indicates an accuracy of predicting the health condition classification using the candidate combination, and the robustness metric of each candidate combination indicates an insensitivity to the manufacturing variabilities on the accuracy.

In order to determine the robustness metric, a variance simulator adds varying percentages of simulated variance to the historical analyte measurements, and the performance of each candidate combination is evaluated with each percentage of the simulated variance. For example, the robustness metric may measure an average percent change in the performance metric per percent change in the simulated variance. In some implementations, the simulated variance is a multiplicative percent bias that is drawn from a normal distribution with a fixed standard deviation and a mean swept between a first, lower percentage and a second, higher percentage. Candidate combinations having high robustness metrics exhibit lower changes in the performance metric per percent change in the simulated variance. The robust combination of features may be selected to effectively balance the performance metric and the robustness metric, such as by selecting the candidate combination that has the highest performance metric after filtering out candidate combinations that have robustness metrics below a threshold.

Once the robust combination of features is selected, a machine learning model is trained to predict the health condition classification based on the robust combination of features extracted from the historical analyte measurements of the user population and the outcome data (e.g., without the simulated variance added). The trained machine learning model processes new analyte measurements of a user collected by the wearable analyte monitoring device over the observation period to predict the health condition classification of the user. In particular, the trained machine learning model processes the robust combination of features extracted from the new analyte measurements of the user to predict the health condition classification.

Broadly speaking, the health condition classification describes a state (or status) of the user during the observation period with respect to the particular health condition classification for which the machine learning model is trained. In some implementations, this “health condition classification” may indicate whether the user has the health condition, is at risk for developing the health condition, and/or indicate adverse effects that the user is predicted to experience. By way of example, the user may have his or her glucose monitored via the analyte monitoring device to predict whether he or she has diabetes (e.g., Type 1 diabetes, Type 2 diabetes, gestational diabetes mellitus (GDM), cystic fibrosis diabetes, and so on), is at risk for developing diabetes (e.g., prediabetes), and/or whether he or she is predicted to experience adverse effects associated with diabetes (e.g., retinopathy, neuropathy, comorbidity, dysglycemia, macrosomia requiring a cesarean section, and neonatal hypoglycemia, to name just a few). In one or more implementations, the machine learning model may additionally or alternatively be configured to predict a type of prediabetes (e.g., impaired glucose fasting (IFG) or impaired glucose tolerance (IGT)). Alternatively or additionally, the health condition classification may correspond to a risk level of having or developing the associated health condition, such as high risk, low risk, or no risk for developing the health condition. In operation, the health condition classification predicted by the machine learning model may be used (e.g., by a health care professional) to treat the person or develop a treatment plan similarly to how the person would be treated if clinically diagnosed using conventional tests (e.g., with a type of diabetes and/or as being susceptible to experiencing adverse effects).

Notably, unlike conventional diabetes tests, the health condition classification (e.g., diabetes classification) predicted by the machine learning model is based on observed analyte (e.g., glucose) values over multiple days. As such, the prediction is more accurate than tests that rely on a blood sample collected at a single point of time. Moreover, unlike the HbA1c test which is an indirect measurement of blood glucose and can be affected by recent changes in glucose levels caused by external factors or conditions such as sickness or pregnancy, the health condition classification predicted by the machine learning model is based on glucose measurements directly obtained during a current observation time period.

The health condition classification prediction is then presented, such as by displaying an indication of the health condition classification to the user, a doctor, or a guardian of the user via a user interface. Other information may also be presented, such as visualizations of the analyte measurements as well as other statistics derived from the analyte measurements. In some cases, the health condition classification prediction is presented in an analyte observation report that may also include one or more treatment options for the user, visual representations of the analyte measurements collected by the analyte monitoring device during the observation period, analyte level statistics of the user generated based on the collected analyte measurements, levels of severity, next steps (e.g., for a doctor, health care professional, or user), a request to follow-up, a request to order more sensors for the analyte monitoring device, activity levels, trends in the analyte level or other markers, patterns in the analyte level or other markers, patterns in exercise, interpretations of the analyte measurements, or activity related to the analyte level. Thus, unlike conventional blood glucose test results for diabetes, the analyte observation report generated by the one or more machine learning models may include a detailed analysis of the prediction as well as various treatment options. It is to be appreciated that health condition classifications and information associated with such classifications may be provided in a variety of ways, including, for example, output as an audio signal via a speaker or digital assistant.

Advantageously, utilizing a wearable analyte monitoring device and a machine learning model that is insensitive to manufacturing variabilities of the analyte monitoring device's sensor to generate predictions of a health condition classification of users increases an accuracy of the predictions and eliminates many of the uncomfortable aspects of the above-noted diagnostic tests and does not limit who can be tested. Unlike HbA1c, for instance, pregnant women can safely wear the analyte monitoring device over the observation period. Moreover, because the machine learning model is applied to a robust combination of features extracted from analyte measurements collected over multiple days, the inconsistencies associated with conventional tests are reduced, thereby increasing the accuracy of the prediction as compared to conventional tests based on a single blood sample. By accurately predicting health condition classifications and notifying users, health care providers, and/or telemedicine services, the described machine learning model allows early detection of the health condition, such as diabetes, and identifies treatment options that may mitigate potentially adverse health conditions. In so doing, serious damage to the heart, blood vessels, eyes, kidneys, and nerves, and death due to diabetes can be largely avoided.

A technical effect of selecting a robust combination of features extracted from analyte measurements based on a performance metric associated with accurately predicting a health condition classification and a robustness metric associated with an insensitivity to manufacturing variabilities of analyte sensors obtaining the analyte measurements is that a machine learning model trained to predict the health condition classification with the robust combination of features may have increased accuracy compared with machine learning models trained with single analyte features or combination of features that are not robust.

In some aspects, the techniques described herein relate to a method, including: obtaining a plurality of features of analyte measurements; selecting a combination of features of the plurality of features based on a robustness metric associated with an insensitivity to manufacturing variabilities of analyte sensors and a performance metric associated with predicting a health condition classification; and training one or more machine learning models to predict the health condition classification using the combination of features.

In some aspects, the techniques described herein relate to a method, wherein the analyte measurements are historical analyte measurements from a user population associated with outcome data of the user population, and wherein selecting the combination of features of the plurality of features based on the robustness metric and the performance metric includes: generating a model prediction of the health condition classification for each of a plurality of candidate combinations of the plurality of features; and determining the performance metric for each of the plurality of candidate combinations based on the model prediction of the health condition classification relative to the outcome data of the user population.

In some aspects, the techniques described herein relate to a method, wherein the performance metric indicates one or both of a sensitivity for predicting the health condition classification and a specificity for predicting the health condition classification based on the model prediction of the health condition classification relative to the outcome data for each of the plurality of candidate combinations.

In some aspects, the techniques described herein relate to a method, wherein the outcome data indicates a clinically determined health condition classification of each user of the user population.

In some aspects, the techniques described herein relate to a method, wherein selecting the combination of features of the plurality of features based on the robustness metric and the performance metric further includes: simulating the manufacturing variabilities of the analyte sensors in the analyte measurements over a plurality of simulation rounds that each introduce a different percentage of simulated variability to the analyte measurements; and determining the robustness metric for each of the plurality of candidate combinations based on a change in the performance metric per percentage of the simulated variability.

In some aspects, the techniques described herein relate to a method, wherein simulating the manufacturing variabilities of the analyte sensors in the analyte measurements over the plurality of simulation rounds includes simulating different performance variabilities and analyte sensor characteristics to introduce the different percentage of simulated variability to the analyte measurements each simulation round.

In some aspects, the techniques described herein relate to a method, wherein selecting the combination of features of the plurality of features based on the robustness metric and the performance metric further includes: filtering the plurality of candidate combinations of the plurality of features based on the robustness metric of each of the plurality of candidate combinations relative to a robustness threshold; and selecting a filtered candidate combination having a highest value for the performance metric as the combination of features.

In some aspects, the techniques described herein relate to a method, wherein the combination of features of the plurality of features includes a first feature and a second feature, and wherein the method further includes determining an individual performance metric and an individual robustness metric for each of the plurality of features using model predictions of the health condition classification for each of the plurality of features.

In some aspects, the techniques described herein relate to a method, wherein at least one of the first feature and the second feature is a trend-related feature of the analyte measurements.

In some aspects, the techniques described herein relate to a method, wherein the second feature is a variability and stability feature of the analyte measurements.

In some aspects, the techniques described herein relate to a method, further including: after training the one or more machine learning models to predict the health condition classification using the combination of features: obtaining new analyte measurements from an analyte measurement device worn by a user over an observation period; extracting features of the combination of features from the new analyte measurements; inputting the extracted features of the combination of features into the one or more machine learning models; and receiving, as an output of the one or more machine learning models, the health condition classification of the user.

In some aspects, the techniques described herein relate to a device including: one or more processors; and a memory having stored thereon computer-readable instructions that are executable by the one or more processors to perform operations including: obtaining analyte data of a user that is measured by an analyte sensor; extracting at least two features of the analyte data, the at least two features included in a multivariate model of analyte features determined to be robust to manufacturing variabilities of the analyte sensor based on variance simulations performed on historical analyte data of a user population; inputting a combination of the at least two features to a machine learning model; predicting, via the machine learning model, a health condition classification of the user; and receiving, as an output of the machine learning model, the health condition classification.

In some aspects, the techniques described herein relate to a device, wherein the analyte data of the user is measured by the analyte sensor over an observation period, and wherein the health condition classification is an indication describing a status of the user during the observation period with respect to a health condition.

In some aspects, the techniques described herein relate to a device, wherein the health condition is diabetes, and wherein the health condition classification is one of a diabetes status, a prediabetes status, and a no diabetes status.

In some aspects, the techniques described herein relate to a device, wherein the machine learning model is trained with training input portions including the combination of the at least two features extracted from the historical analyte data of the user population and expected output portions including labels representative of the health condition classification of each user of the user population.

In some aspects, the techniques described herein relate to a system including: a wearable analyte monitoring device including a sensor that is inserted subcutaneously into skin of a user to collect analyte measurements of the user during an observation period; a storage device to maintain the analyte measurements of the user collected during the observation period; and a prediction system to predict a health condition classification of the user by extracting a robust analyte feature combination from the analyte measurements of the user and processing the robust analyte feature combination using one or more machine learning models.

In some aspects, the techniques described herein relate to a system, wherein the one or more machine learning models are generated based on historical analyte measurements and historical outcome data of a user population, and the system further includes a model manager to: obtain the historical analyte measurements and the historical outcome data of the user population, the historical analyte measurements provided by analyte monitoring devices worn by users of the user population; extract the robust analyte feature combination from the historical analyte measurements; and generate the one or more machine learning models by: providing the robust analyte feature combination extracted from the historical analyte measurements to the one or more machine learning models; and adjusting weights of the one or more machine learning models based on a comparison of training health condition classifications received from the one or more machine learning models and clinically verified health condition classifications indicated by the historical outcome data.

In some aspects, the techniques described herein relate to a system, wherein the clinically verified health condition classifications indicated by the historical outcome data are associated with one or more diagnostic measures independent of the historical analyte measurements provided by the analyte monitoring devices worn by users of the user population.

In some aspects, the techniques described herein relate to a system, wherein one or both of the storage device and the prediction system is implemented, at least in part, at the wearable analyte monitoring device.

In some aspects, the techniques described herein relate to a system, wherein the prediction system is implemented at one or more computing devices remote from the wearable analyte monitoring device.

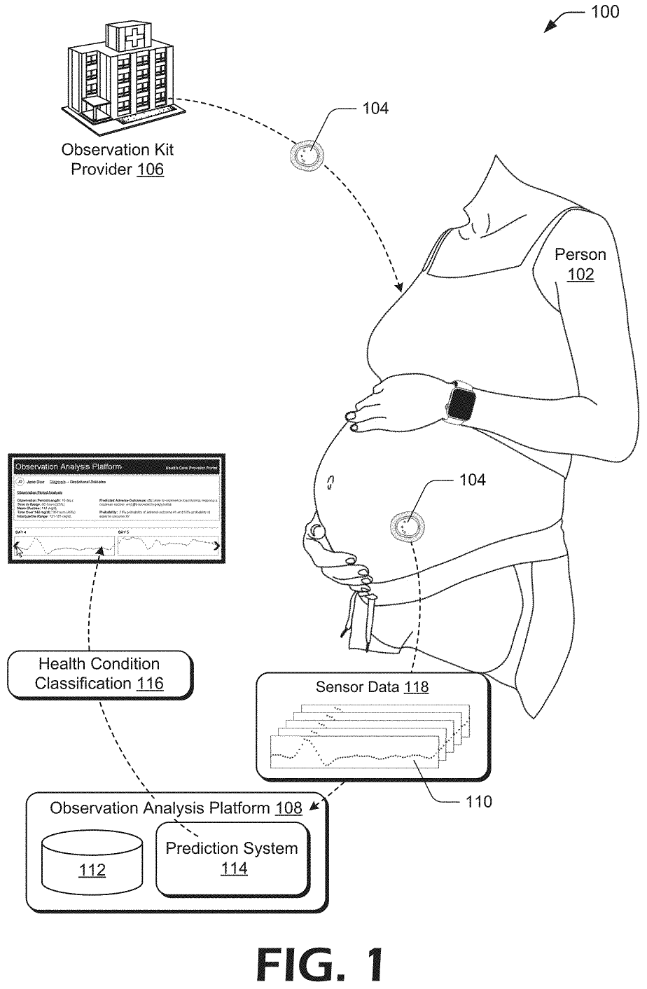

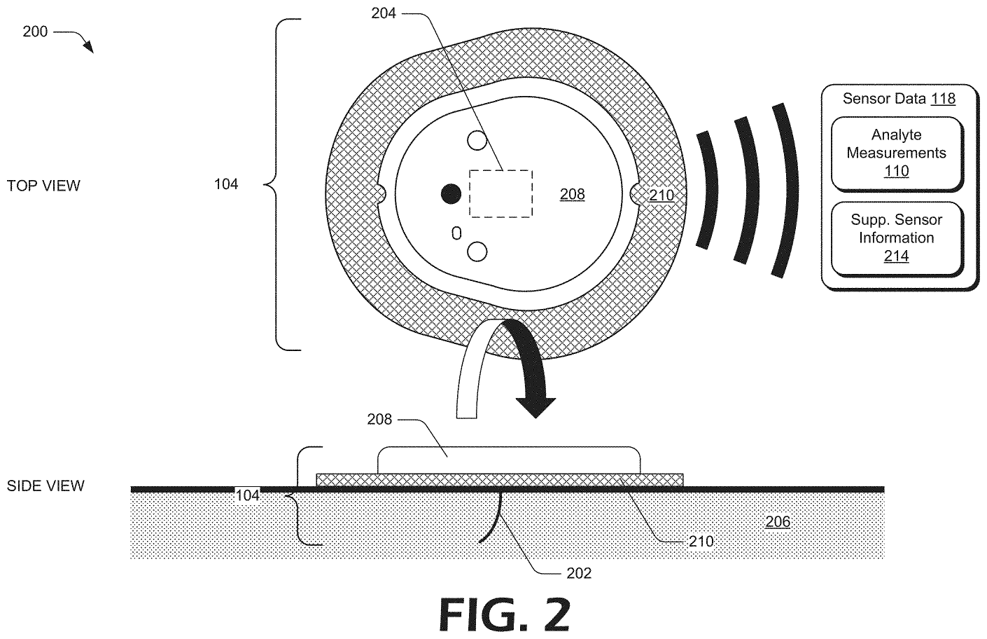

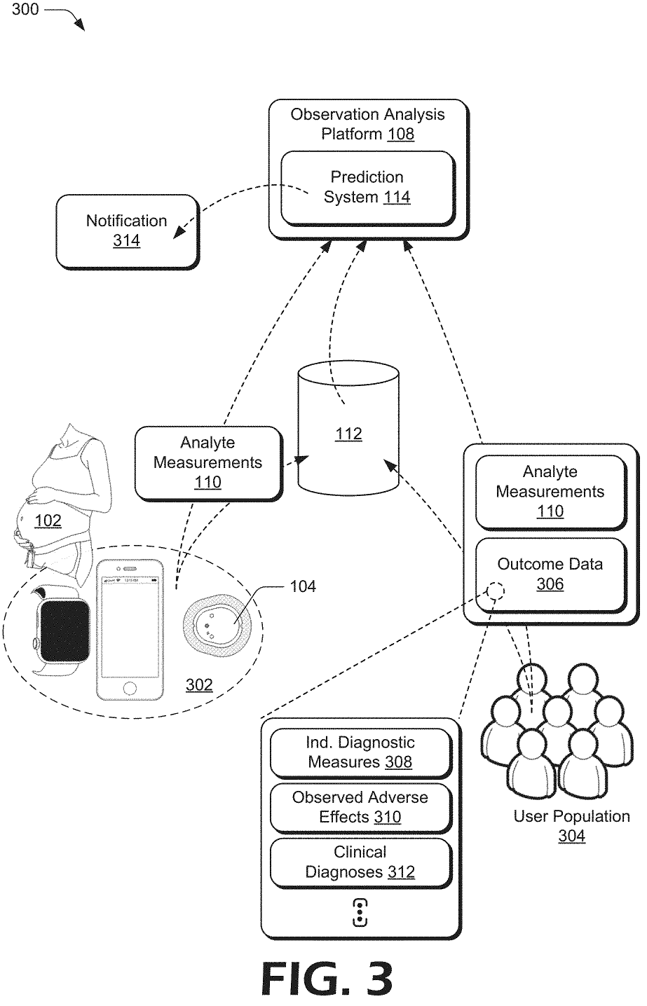

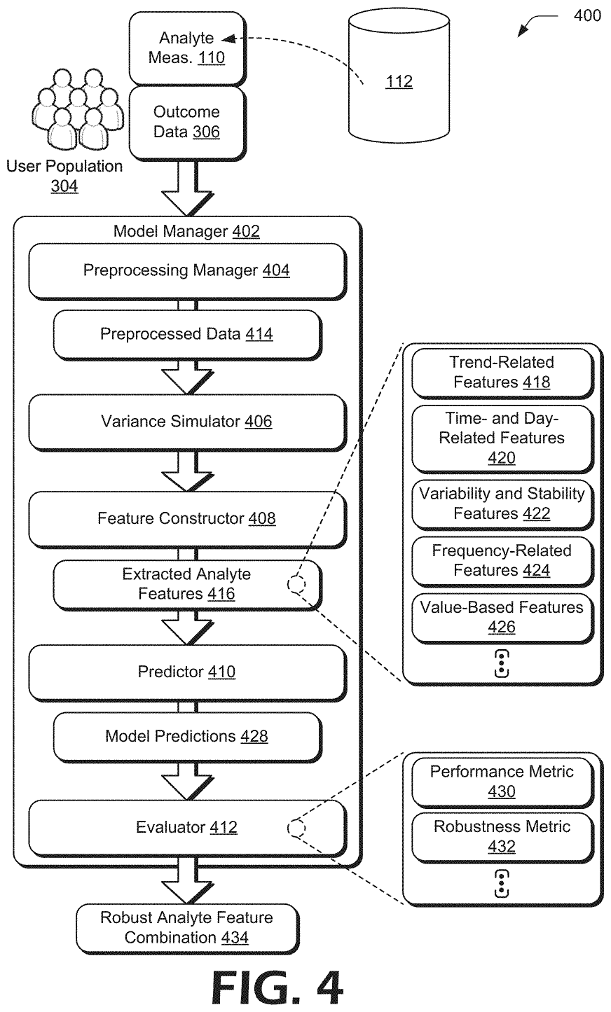

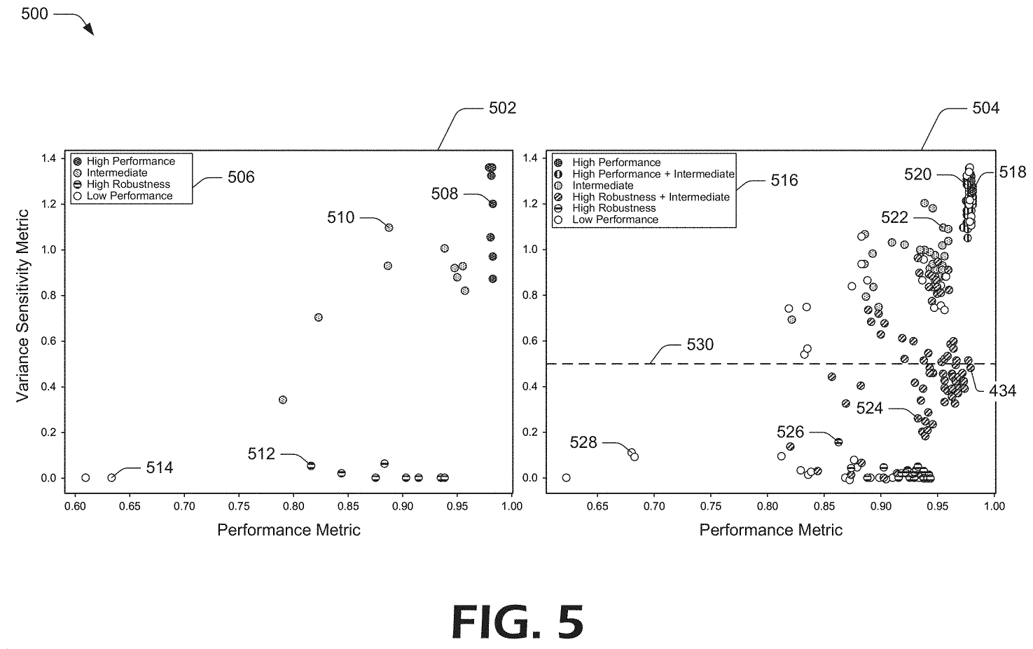

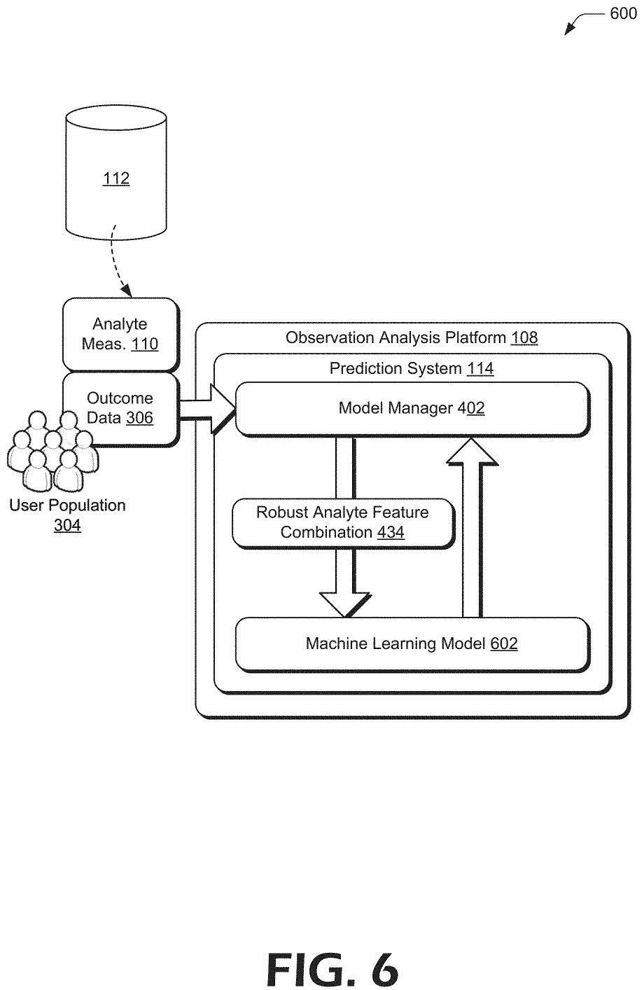

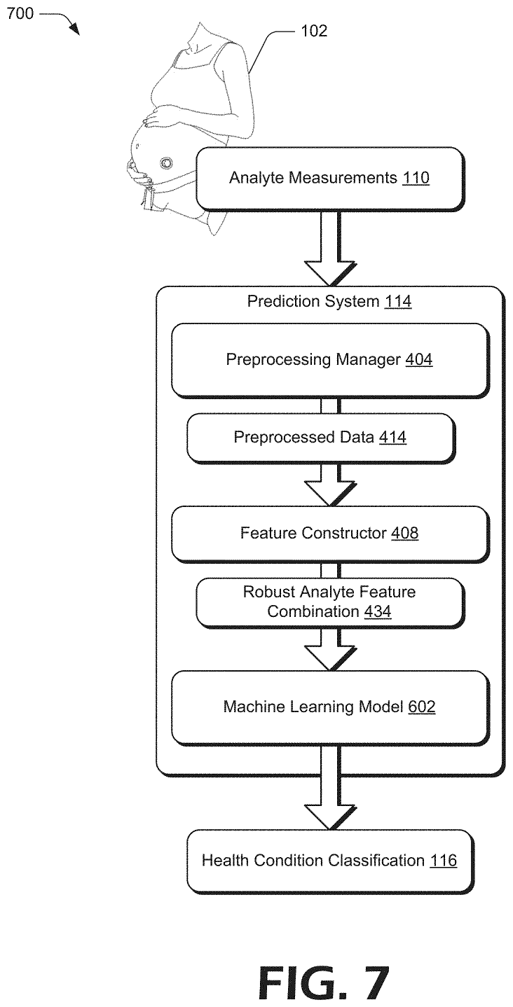

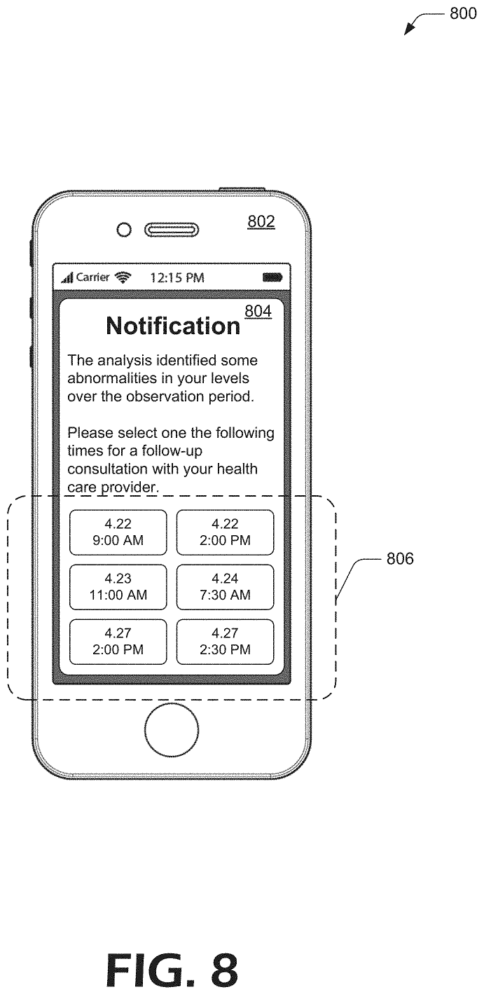

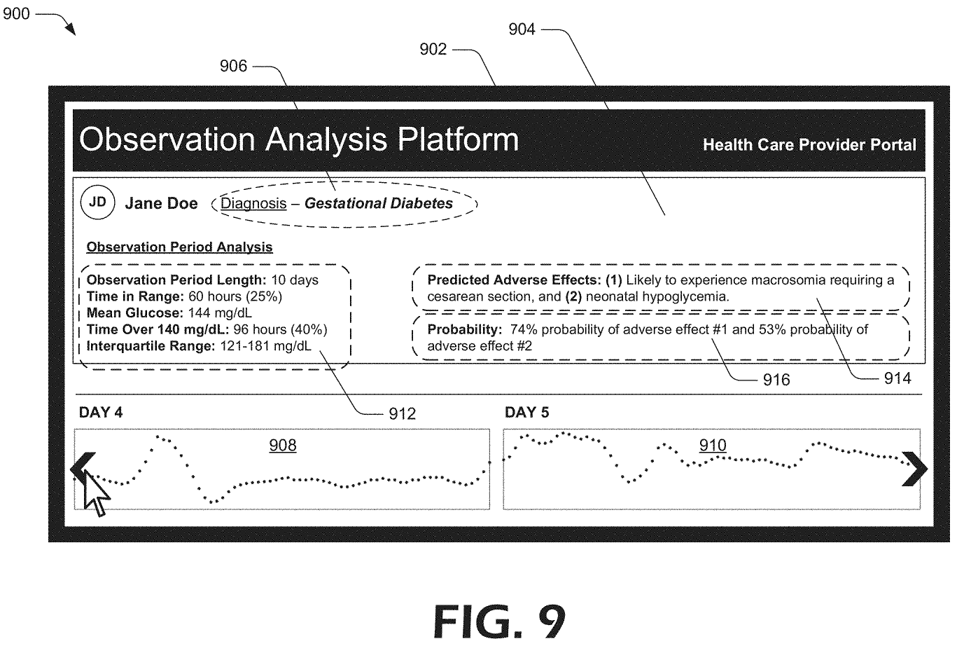

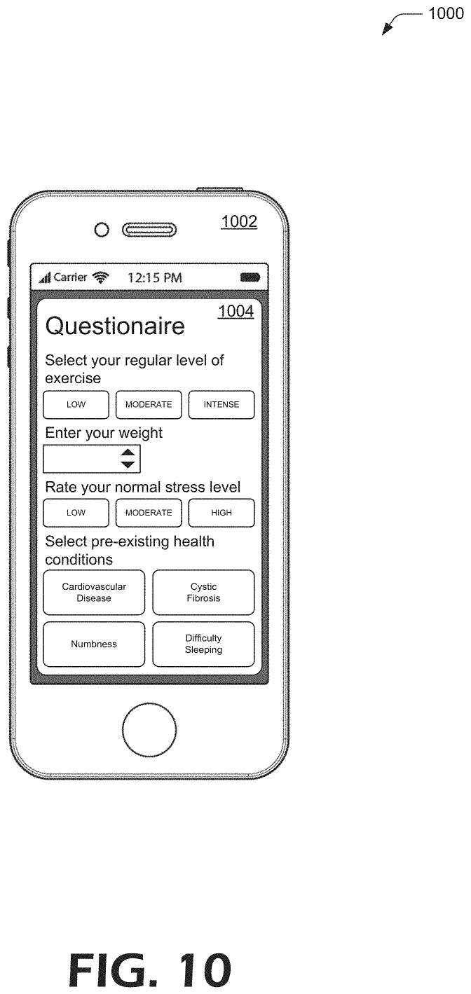

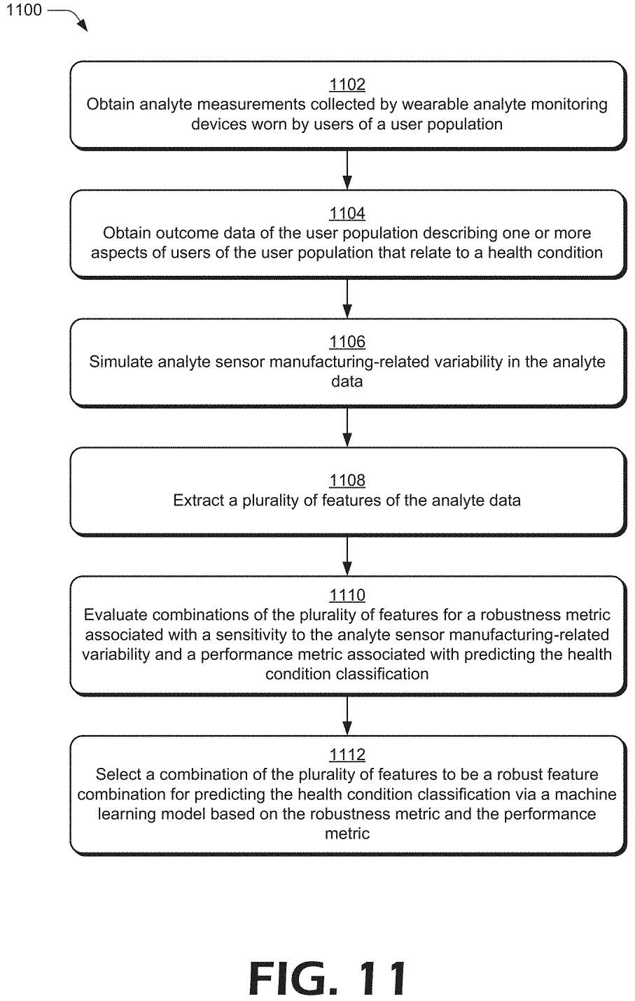

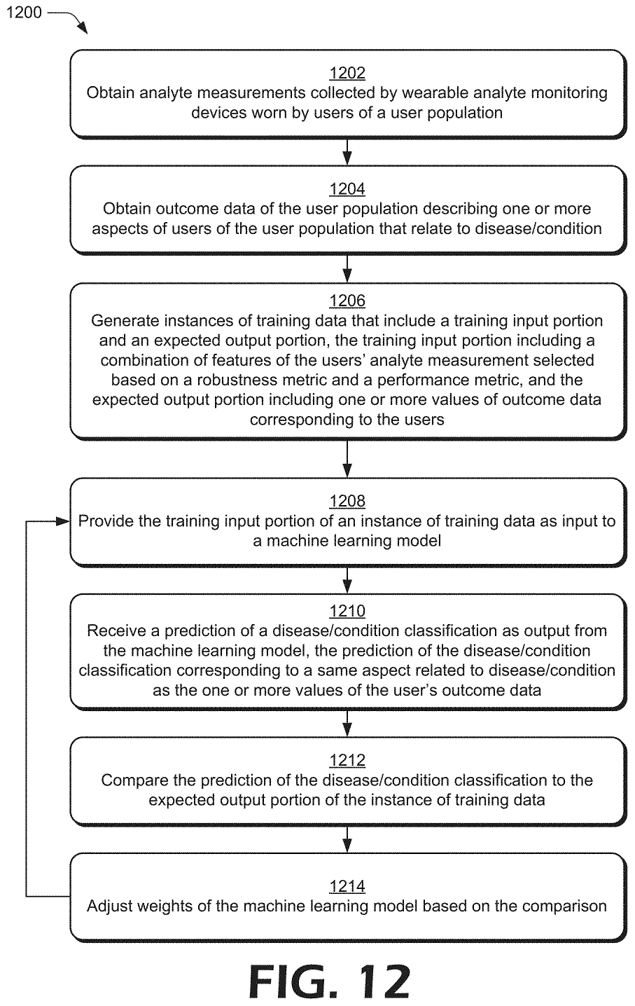

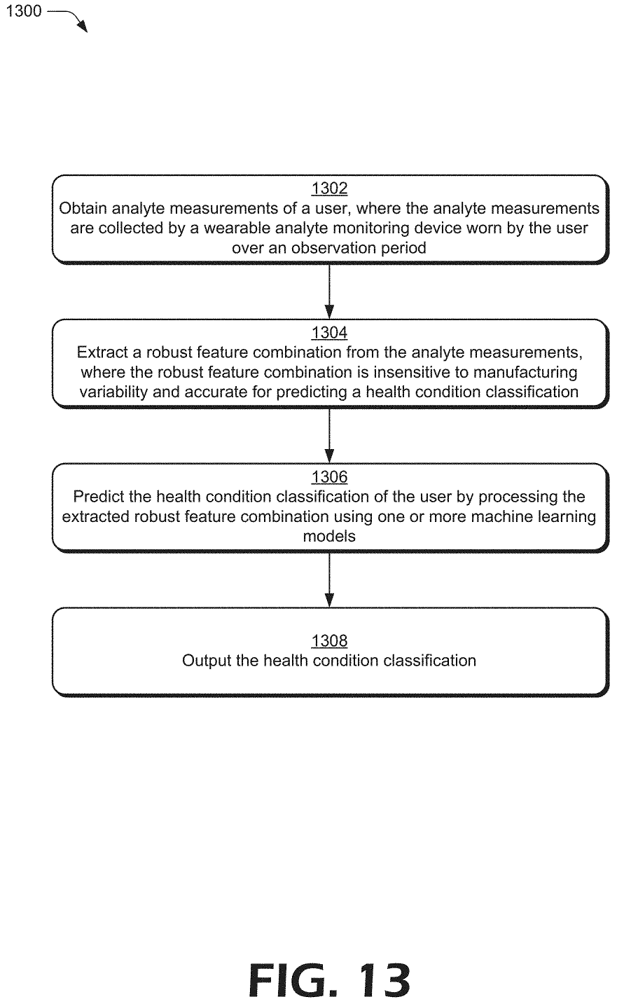

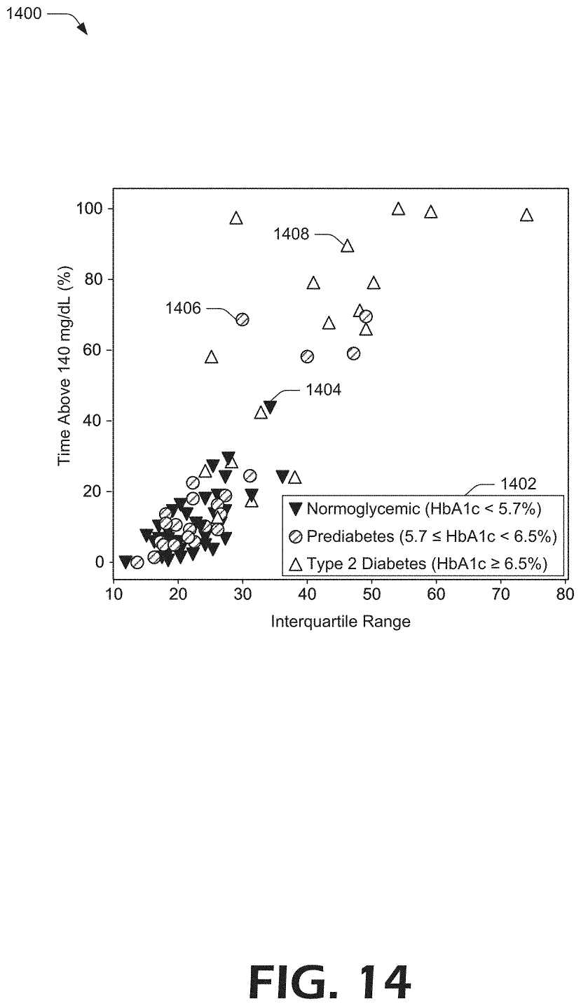

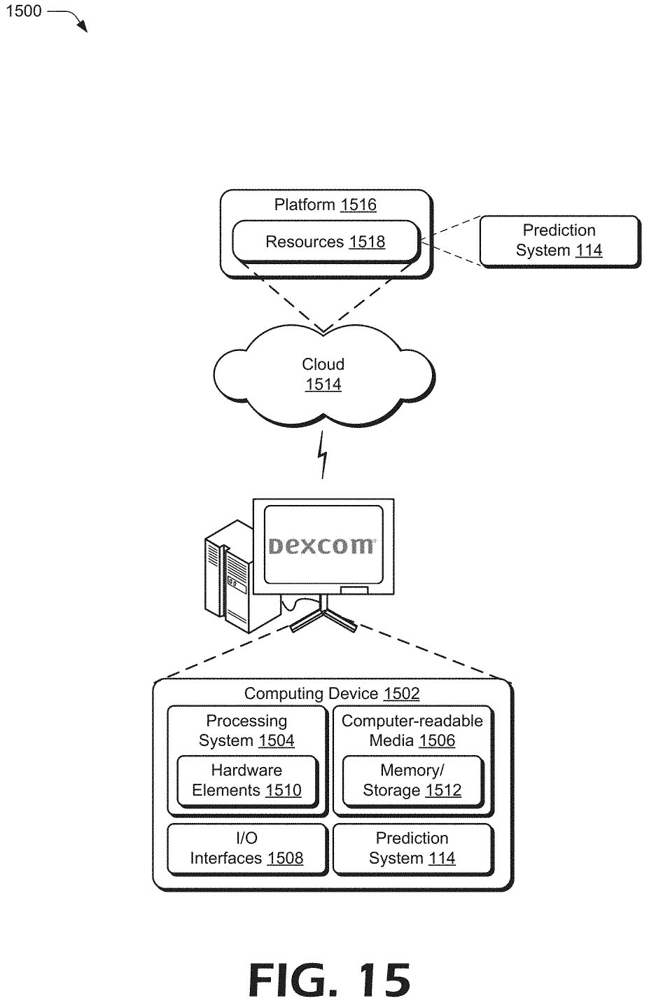

In the following discussion, an example of an environment is first described that may employ the techniques described herein. For example, an analyte monitoring device may be used to measure one or more analytes from a user over an observation period, such as according to the example implementation shown in FIG. 1. The analyte monitoring device may employ a sensor, such as shown in FIG. 2, to measure the one or more analytes. In accordance with the techniques described herein, analyte data generated by the analyte monitoring device may be routed to different systems in connection with health condition classification, such as illustrated in FIG. 3. Examples of implementation details and procedures are then described which may be performed in the discussed environment as well as other environments. For example, as illustrated in the implementation of FIG. 4 and the procedure (e.g., method) of FIG. 11, a model manager may perform a variance simulation on historical analyte measurements of a user population to identify a combination of analyte features that produces accurate health condition predictions even in the presence of the simulated variance. Example graphs showing performance metrics and variance sensitivity metrics for a plurality of analyte features alone and in combination are depicted in FIG. 5. Once the combination of analyte features is selected, the model manager may train a machine learning model to predict a health condition classification with the combination of analyte features of the historical analyte measurements and outcome data of the user population, such as illustrated in the implementation of FIG. 6 and the procedure of FIG. 12. Once trained, the machine learning model may be used to predict the health condition classification of a user based on the combination of analyte features extracted from analyte data of the user, such as according to the implementation shown in FIG. 7 and the procedure of FIG. 13. The health condition classification may be input via the example user interfaces shown in FIGS. 8 and 9. Further, additional health data that may be used in predicting the health condition classification may be input via the example user interface of FIG. 10. One example of how metrics derived from glucose measurements may be used in a diabetes classification algorithm is discussed with respect to FIG. 14. FIG. 15 shows an example system including various components that can be implemented as any type of computing device for implementing and performing the above-described techniques. Performance of those procedures is not limited to the example of the environment and the example of the environment is not limited to performance of those procedures.

Example Environment

FIG. 1 is an illustration of an environment 100 in an example of an implementation that is operable to employ health condition prediction using analyte measurements and machine learning as described herein. The illustrated environment 100 includes a person 102, who is depicted wearing an analyte monitoring device 104. The illustrated environment also includes an observation kit provider 106 and an observation analysis platform 108.

In the illustrated environment 100, the analyte monitoring device 104 is depicted being provided by the observation kit provider 106 to the person 102, e.g., as part of an observation kit. The analyte monitoring device 104 may be provided as part of an observation kit, for instance, for the purpose of monitoring one or more analytes in the person 102's blood and/or interstitial fluid over an observation period lasting multiple days or lasting a different amount of time, e.g., minutes, hours, and so forth. As used herein, the term “analyte” may refer to a biochemical or chemical substance that is subjected to analysis in determining a state of a user with respect to a health condition. For example, the term “analyte” may refer to a metabolite or other chemical, a protein (e.g., an enzyme), or another measurable indicator of a severity or presence of the health condition (or an absence thereof) that is present in blood and/or interstitial fluid. For example, the analyte monitoring device 104 may be configured to measure one or more of glucose, lactate, ketones, potassium, insulin, phosphate, bicarbonate, calcium, magnesium, sodium, and blood urea nitrogen. By way of example, the person 102 may have his or her glucose monitored to predict whether he or she has diabetes (e.g., Type 1 diabetes, Type 2 diabetes, gestational diabetes mellitus (GDM), cystic fibrosis diabetes, and so on), is at risk for developing diabetes (e.g., prediabetes), and/or is predicted to experience adverse effects associated with diabetes (e.g., comorbidity, dysglycemia, macrosomia requiring a cesarean section (C-section), and neonatal hypoglycemia, to name just a few). As another example, the analyte monitoring device 104 may monitor one or more analytes to predict whether the person 102 has an acute condition (e.g., ketonemia, acidosis, or sepsis) or a chronic condition (e.g., liver disease, kidney disease, or sleep apnea).

In connection with the observation period, instructions may be provided to the person 102 that instruct the person 102 to perform one or more activities during the observation period, such as instructing the person 102 to consume a beverage or specific meal (e.g., a same beverage as is consumed in connection with OGTT), avoid one or more specific foods, exercise, and rest, to name just a few. In one or more implementations, the instructions may be provided as part of an observation kit, e.g., written instructions. Alternatively or additionally, the observation analysis platform 108 may cause instructions to be communicated to and output (e.g., for display or audio output) via the one or more computing devices associated with the person 102. The observation analysis platform 108 may provide these instructions for output after a predetermined amount of time in the observation period has lapsed (e.g., two days) and/or based on patterns in the analyte measurements obtained. In connection with providing such instructions, the analyte monitoring device 104 automatically monitors the person 102's analyte level after performance of the instructed activity, such as by monitoring an amount the person 102's glucose changes after consuming the meal instructed, performing the exercise instructed, and so forth.

Although discussed throughout as lasting multiple days, in one or more implementations, a duration of the observation period may be variable, such that when enough analyte measurements have been collected to accurately predict a health condition classification for the person 102, the observation period may end. As an example, in some cases, the person 102's glucose measurements over just a few hours may be processed to predict the person 102 has diabetes with statistical certainty. In this case, the duration of the observation period may be a number of hours rather than multiple days. In general, though, the observation period lasts multiple days to obtain data so that features can be extracted to describe, for example, day-over-day variations in analyte levels and to prevent erroneous predictions that may be otherwise caused by anomalous measurements or observations.

To this end, the observation kit provider 106 may represent one or more of a variety of entities associated with obtaining a prediction regarding whether the person 102 has a health condition or is predicted to experience adverse effects of the health condition. For instance, the observation kit provider 106 may represent a provider of the analyte monitoring device 104 and of a platform that monitors and analyzes measurements obtained therefrom, such as the observation analysis platform 108 when it also corresponds to the provider of the analyte monitoring device 104. Alternatively or additionally, the observation kit provider 106 may correspond to a health care provider (e.g., a primary care physician, OB/GYN, endocrinologist), a doctor's office, a hospital, an insurance provider, a medical testing laboratory, or a telemedicine service, to name just a few. Alternatively or additionally, the observation kit provider 106 may correspond to a pharmacist or pharmacy, which may have a physical brick-and-mortar location and/or provide service online. It is to be appreciated that these are just a few examples, and the observation kit provider 106 may represent different entities without departing from the spirit or scope of the described techniques.

Given this, provision of the analyte monitoring device 104 to the person 102 may occur in various ways in accordance with the described techniques. For example, the analyte monitoring device 104 may be handed to the person 102 at a doctor's office, hospital, medical testing laboratory, or a brick-and-mortar pharmacy, e.g., as part of an observation kit. Alternatively, the analyte monitoring device 104 may be mailed to the person 102, e.g., from the provider of the analyte monitoring device 104, a pharmacy, a medical testing laboratory, a telemedicine service, and so forth. The person 102 may obtain the analyte monitoring device 104 for an observation period in other ways in one or more implementations.

Regardless of how the analyte monitoring device 104 is obtained by the person 102, the analyte monitoring device 104 is configured to monitor one or more pre-determined analytes (e.g., glucose) of the person 102 during an observation period, which lasts for a time period spanning multiple days. In one or more implementations, the analyte monitoring device 104 is a wearable device, such that it is worn by the person 102 while the device performs various operations. Additionally or alternatively, the analyte monitoring device 104 performs one or more operations before or after being worn by the person 102. For example, the analyte monitoring device 104 may be configured with an analyte sensor that detects one or more signals indicative of the analyte in the person 102 and enables generation of analyte measurements or estimations (e.g., estimated glucose values). Those analyte measurements (e.g., glucose measurements) may correspond to or otherwise be packaged for communication to one or more of the computing devices or the observation analysis platform 108 as analyte measurements 110, which is one example of sensor data 118.

In at least one implementation, the analyte monitoring device 104 is a glucose monitoring system. As an example, the analyte monitoring device 104 may be configured as a continuous glucose monitoring (“CGM”) system, e.g., a wearable CGM system. As used herein in connection with analyte monitoring, the term “continuous” may refer to an ability of a device to produce measurements substantially continuously, such that the device may be configured to produce the analyte measurements at regular or irregular intervals of time (e.g., approximately every hour, approximately every 30 minutes, approximately every 5 minutes, and so forth), responsive to establishing a communicative coupling with a different device (e.g., when the observation analysis platform 108 establishes a wireless connection with the analyte monitoring device 104 to retrieve one or more of the measurements), and so forth. In other implementations, however, the glucose (or other analyte) monitoring may not be “continuous”, and instead provides glucose measurements when requested. For example, the analyte monitoring device 104 may communicate a current glucose measurement to a computing device responsive to a request from the computing device. This request may be initiated in a variety of different ways, such as responsive to user input to a user interface displayed by the computing device, responsive to placement of the computing device within a threshold proximity to the analyte monitoring device 104, responsive to the computing device making physical contact with the analyte monitoring device 104, responsive to a request from an application implemented at the computing device, and so forth. This functionality along with further aspects of the configuration of the analyte monitoring device 104 are discussed in more detail in relation to FIG. 2.

In addition to producing sensor data 118 (including the analyte measurements 110), the analyte monitoring device 104 also transmits the produced sensor data 118, e.g., to the observation analysis platform 108. The analyte monitoring device 104 may communicate the data in real-time (e.g., as it is produced) using an analyte sensor or other sensors. Alternatively or in addition, the analyte monitoring device 104 may communicate the data to the observation analysis platform 108 at predefined intervals of time. For example, the analyte monitoring device 104 may be configured to communicate the sensor data 118 to the observation analysis platform 108 approximately every five minutes. An interval at which the sensor data 118 is communicated by the analyte monitoring device 104 may be different from the examples above without departing from the spirit or scope of the described techniques. The sensor data 118 may be communicated by the analyte monitoring device 104 to other computing devices (e.g., in addition to or as an alternative to the observation analysis platform 108 in accordance with the described techniques.

Although the analyte monitoring device 104 may be configured in a similar manner as wearable analyte monitoring devices used for treating the health condition, such as for treating diabetes, in one or more implementations, the analyte monitoring device 104 may be configured differently than the devices used for treatment. These different configurations may be deployed to control confounding factors of observation periods so that measurements are obtained that accurately reflect the effects of users' normal, day-to-day behavior. This can include, for instance, limiting and/or completely preventing users from inspecting those measurements during the observation period. By preventing users from inspecting the analyte measurements 110 over the course of observation periods, the observation configurations further prevent users from seeing or otherwise observing analyte measurement events (e.g., spikes in glucose) and changing their behavior to counteract such events.

In some cases, the analyte monitoring device 104 may be a specialized device designed specifically for the purpose of collecting glucose measurements for a user during an observation period spanning multiple days or some other period of time so that a diabetes classification may be generated, which may be differentiated in one or more ways from wearable glucose monitoring devices worn by users to treat diabetes. In other instances, the analyte monitoring device 104 may have the same hardware characteristics as the wearable glucose monitoring devices used to treat diabetes but may include software that disables or enables different functionality, such as software that prevents the user from inspecting analyte measurements 110 during the observation period. In these instances, functionality that is disabled during the observation period can be enabled after the observation period has ended so that the user has access to the previously-disabled functionality, such as the ability to view glucose measurements in substantially real-time.

The different configurations also may be based on differences between how the analyte measurements 110 are used in connection with an observation period for health condition prediction and how measurements are used in connection with treatment of the health condition. In an example where the health condition is diabetes and the analyte is glucose, with treatment, continuous or nearly continuous receipt and output of glucose measurements, substantially as those measurements are produced, may be used to inform treatment decisions, e.g., to help a person or his or her caretaker decide what to eat, how to administer insulin, whether to contact a health care provider, and so on. In those scenarios, knowing the measurements and/or trends of the measurements in a timely manner (e.g., in substantially real-time) may be critical to effectively mitigating potentially severe adverse effects. By way of contrast, receipt and substantially real-time output of glucose measurements to a person being observed (or to a caretaker) may be unnecessary in connection with diabetes prediction in these scenarios. Instead, the glucose measurements produced for diabetes prediction are handled so that at the end of the observation period, or after some other horizon (e.g., when enough measurements have been produced to achieve statistical certainty), an accurate prediction regarding diabetes can be generated.

Based on such differences with respect to how the analyte measurements are used, the analyte monitoring device 104 may have more local storage than wearable analyte measurement devices used for treatment (e.g., diabetes treatment). By way of example and not limitation, 10-15 days' worth of analyte measurement storage may be provided for observation configurations, whereas 3 hours' worth of analyte measurement storage may be provided for treatment configurations. The larger storage capacity of the analyte monitoring device 104 may be suitable to store the analyte measurements 110 for the duration of the observation period. In contrast, wearable analyte measurement devices used for treatment may be configured to offload analyte measurements such that once the measurements are suitably offloaded, they are no longer stored locally on those devices. By way of example, wearable glucose devices used for treatment of diabetes may offload glucose measurements by transmitting them via wireless connections to an external computing device, e.g., at predetermined time intervals and/or responsive to establishing or reestablishing a connection with the computing device.

To the extent that the analyte monitoring device 104 may be configured to store the analyte measurements 110 for an entirety of an observation period, in one or more implementations, the analyte monitoring device 104 may be configured without wireless transmission means, e.g., without any antennae to transmit the analyte measurements 110 wirelessly and without hardware or firmware to generate packets for such wireless transmission. Instead, the analyte monitoring device 104 may be configured with hardware to communicate the analyte measurements 110 via a physical, wired coupling. In such scenarios, the analyte monitoring device 104 may be “plugged in” to extract the analyte measurements 110 from the device's storage.

Accordingly, the analyte monitoring device 104 may be configured with one or more ports to enable wired transmission of the analyte measurements 110 to an external computing device. Examples of such physical couplings may include micro universal serial bus (USB) connections, mini-USB connections, and USB-C connections, to name just a few. Although the analyte monitoring device 104 may be configured for extraction of the analyte measurements 110 via wired connections as discussed just above, in different scenarios, the analyte monitoring device 104 may be alternatively or additionally configured to offload the analyte measurements 110 over one or more wireless connections. Implementations involving wired and/or wireless communication of the analyte measurements 110 are discussed further below.

In addition to storage and communication differences, the analyte monitoring device 104 may also include one or more sensors or sensor circuitry configured differently than in devices designed for treatment of the health condition. For instance, sensors and the circuitry (e.g., including measurement algorithms) of wearable glucose monitoring devices used for treating diabetes may be optimized for a range of measurements spanning from 40 mg/dL to 400 mg/dL. This is because treatment of diabetes often involves deciding what actions to take to mitigate severe glycemic events that can occur toward ends of the range, e.g., hypo- and hyper-glycemia. To predict diabetes, however, fidelity of the measurements over as wide a range may not be needed. Rather, diabetes predictions may be suitably generated in relation to a smaller range, such as a range of glucose measurements spanning from 120 mg/dL to 240 mg/dL. Accordingly, the analyte monitoring device 104 may include one or more sensors or sensor circuitry optimized to produce measurements in such a smaller range. It is to be appreciated that the above-discussed differences are merely examples of how the analyte monitoring device 104 may differ from wearable analyte monitoring devices configured for treatment of the health condition and that the analyte monitoring device 104 may differ from those devices in different ways without departing from the spirit or scope of the described techniques.

Once the analyte monitoring device 104 produces the analyte measurements 110, the measurements are provided to the observation analysis platform 108. As noted above, the analyte measurements 110 may be communicated to the observation analysis platform 108 as the sensor data 118 over wired and/or a wireless connection. In scenarios where the observation analysis platform 108 is implemented partially or entirely on the analyte monitoring device 104, for instance, the analyte measurements 110 may be transferred over a bus from the device's local storage to a processing system of the device. In scenarios where the analyte monitoring device 104 is configured to generate a prediction of a health condition classification by processing the analyte measurements 110, the analyte monitoring device 104 may also be configured to provide the predicted health condition classification as output, e.g., by communicating the health condition classification to an external computing device. In other scenarios, the analyte measurements 110 may be processed by an external computing device configured to predict health condition classifications.

In one or more implementations, the analyte monitoring device 104 is configured to transmit the analyte measurements 110 to an external device over a wired connection with the external device, e.g., via USB-C or some other physical, communicative coupling. Here, a connector may be plugged into the analyte monitoring device 104, or the analyte monitoring device 104 may be inserted into an apparatus having a receptacle that interfaces with corresponding contacts of the device. The analyte measurements 110 may then be obtained from storage of the analyte monitoring device 104 via this wired connection, e.g., transferred over the wired connection to the external device. Such a connection may be used in scenarios where the analyte monitoring device 104 is mailed by the person 102 after the observation period, such as to a health care provider, telemedicine service, provider of the analyte monitoring device 104, or medical testing laboratory. To this end, an observation kit (not shown) may include packaging (e.g., an envelope or box) to mail the analyte monitoring device 104 to such an entity after observation. Such a connection may also be used in scenarios where the analyte monitoring device 104 is dropped off by the person 102 after the observation period, such as at a doctor's office or hospital (or other establishment of a health care provider), a pharmacy, or a medical testing laboratory. Alternatively or additionally, scenarios involving a wired connection may involve the person 102 plugging in the analyte monitoring device 104 to an external computing device after the testing period, e.g., using a cord provided as part of an observation kit. In these scenarios, the external computing device may communicate the analyte measurements 110 to the observation analysis platform 108 over a network (not shown), such as the Internet.

Alternatively or additionally, provision of the analyte measurements 110 to the observation analysis platform 108 may involve the analyte monitoring device 104 communicating the analyte measurements 110 over one or more wireless connections. For example, the analyte monitoring device 104 may wirelessly communicate the analyte measurements 110 to external computing devices, such as a mobile phone, tablet device, laptop, smart watch, other wearable health tracker, and so on. Accordingly, the analyte monitoring device 104 may be configured to communicate with external devices using one or more wireless communication protocols or techniques. By way of example, the analyte monitoring device 104 may communicate with external devices using one or more of Bluetooth (e.g., Bluetooth Low Energy links), near-field communication (NFC), Long Term Evolution (LTE) standards such as 5G, and so forth. The analyte monitoring devices 104 may be configured with corresponding antennae and other wireless transmission means in scenarios where the analyte measurements 110 are communicated to an external device for processing. In those scenarios, the analyte measurements 110 may be communicated to the observation analysis platform 108 in various manners, such as at predetermined time intervals (e.g., every day, every hour, or every five minutes), responsive to occurrence of some event (e.g., filling a storage buffer of the analyte monitoring device 104), or responsive to an end of an observation period, to name just a few.

Thus, regardless of where the observation analysis platform 108 is implemented, the observation analysis platform 108 obtains the analyte measurements 110 produced by the analyte monitoring device 104. In one or more implementations, the observation analysis platform 108 may be implemented in whole or in part at the analyte monitoring device 104. Alternatively or additionally, the observation analysis platform 108 may be implemented in whole or in part using one or more computing devices external to the analyte monitoring device 104, such as one or more computing devices associated with the person 102 (e.g., a mobile phone, tablet device, laptop, desktop, or smart watch) or one or more computing devices associated with a service provider (e.g., a health care provider, a telemedicine service, a service corresponding to the provider of the analyte monitoring device 104, a medical testing laboratory service, and so forth). In the latter scenario, the observation analysis platform 108 may be implemented at least in part on one or more server devices.

In the illustrated environment 100, the observation analysis platform includes a storage device 112. In accordance with the described techniques, the storage device 112 is configured to maintain the analyte measurements 110. The storage device 112 may represent one or more databases and other types of storage capable of storing the analyte measurements 110. The storage device 112 may also store a variety of other data, such as demographic information describing the person 102, information about a health care provider, information about an insurance provider, payment information, prescription information, determined health indicators, account information (e.g., username and password), and so forth. As discussed in more detail below, the storage device 112 may also maintain data of other users of a user population.

In the illustrated environment 100, the observation analysis platform 108 also includes prediction system 114. The prediction system 114 represents functionality to process the analyte measurements 110 to generate disease and condition predictions, such as to predict whether the person 102 has diabetes (e.g., Type 2 diabetes, GDM, cystic fibrosis diabetes, and so on), is at risk for developing diabetes (e.g., prediabetes), and/or is predicted to experience adverse effects associated with diabetes (e.g., comorbidity, dysglycemia, macrosomia requiring a C-section, and neonatal hypoglycemia, to name just a few). As discussed in more detail below, the prediction system 114 uses machine learning to predict health condition classifications. Use of machine learning may include, for instance, leveraging one or more models generated using machine learning techniques as well as using historical analyte measurements and historical outcome data of a user population to identify robust features of the analyte measurements to use for predicting the health condition classifications.

The illustrated environment 100 also includes health condition classification 116, which may be output by the prediction system 114. In accordance with the described techniques, the health condition classification 116 may indicate whether it is predicted the person has an indicated health condition or is predicted to experience adverse effects associated with the health condition. The health condition classification 116 may also be used to generate one or more notifications or user interfaces based on the classification, such as a report directed to a health care provider that includes the health condition classification (e.g., that the person is predicted to have the health condition, such as diabetes) or a notification directed to the person 102 that instructs the person 102 to contact his or her health care provider. Examples of user interfaces that may be generated based on the health condition classification 116 are described in more detail in relation to FIGS. 8 and 9. In the context of measuring analytes, such as analyzing one or more analytes of interest continuously, and obtaining data describing such measurements, consider the following discussion of FIG. 2.

FIG. 2 depicts an example 200 of an implementation of the analyte monitoring device 104 of FIG. 1 in greater detail. As such, components previously introduced in FIG. 1 are numbered the same and will not be re-introduced. In particular, the illustrated example 200 includes a top view and a corresponding side view of the analyte monitoring device 104. It is to be appreciated that the analyte monitoring device 104 may vary in implementation from the following discussion in various ways without departing from the spirit or scope of the described techniques.

In this example 200, the analyte monitoring device 104 is illustrated to include an analyte sensor 202 (e.g., a glucose sensor) and a sensor module 204. Here, the analyte sensor 202 is depicted in the side view having been inserted subcutaneously into skin 206, e.g., of the person 102 of FIG. 1. The sensor module 204 is approximated in the top view as a dashed rectangle. The analyte monitoring device 104 also includes a transmitter 208 in the illustrated example 200. Use of the dashed rectangle for the sensor module 204 indicates that it may be housed or otherwise implemented within a housing of the transmitter 208. Antennae and/or other hardware used to enable the transmitter 208 to produce signals for communicating data, e.g., over a wireless connection to the observation analysis platform 108, may also be housed or otherwise implemented within the housing of the transmitter 208. In this example 200, the analyte monitoring device 104 further includes an adhesive pad 210.

In operation, the analyte sensor 202 and the adhesive pad 210 may be assembled to form an application assembly, where the application assembly is configured to be applied to the skin 206 so that the analyte sensor 202 is subcutaneously inserted as depicted. In such scenarios, the transmitter 208 may be attached to the assembly after application to the skin 206 via an attachment mechanism (not shown). Alternatively, the transmitter 208 may be incorporated as part of the application assembly, such that the analyte sensor 202, the adhesive pad 210, and the transmitter 208 (with the sensor module 204) can all be applied at once to the skin 206. In one or more implementations, this application assembly is applied to the skin 206 using a separate sensor applicator (not shown). Unlike the finger sticks required by conventional blood glucose meters, for example, user-initiated application of the analyte monitoring device 104 with a sensor applicator is nearly painless and does not require the withdrawal of blood. Moreover, the automatic sensor applicator generally enables the person 102 to embed the analyte sensor 202 subcutaneously into the skin 206 without the assistance of a clinician or health care provider.

The analyte monitoring device 104 may also be removed by peeling the adhesive pad 210 from the skin 206. It is to be appreciated that the analyte monitoring device 104 and its various components are illustrated as one example form factor, and the analyte monitoring device 104 and its components may have different form factors without departing from the spirit or scope of the described techniques.

In operation, the analyte sensor 202 is communicably coupled to the sensor module 204 via at least one communication channel, which can be a wireless connection or a wired connection. Communications from the analyte sensor 202 to the sensor module 204 or from the sensor module 204 to the analyte sensor 202 can be implemented actively or passively, and these communications can be continuous (e.g., analog) or discrete (e.g., digital).

The analyte sensor 202 may be a device, a molecule, and/or a chemical that changes or causes a change in response to an event which is at least partially independent of the analyte sensor 202. The sensor module 204 is implemented to receive indications of changes to the analyte sensor 202 or caused by the analyte sensor 202. For example, the analyte sensor 202 can include glucose oxidase, which reacts with glucose and oxygen to form hydrogen peroxide that is electrochemically detectable by the sensor module 204, which may include an electrode. In this example, the analyte sensor 202 may be configured as or may include a glucose sensor configured to detect analytes in blood or interstitial fluid that are indicative of a glucose level using one or more measurement techniques. In one or more implementations, the analyte sensor 202 may also be configured to detect analytes in the blood or the interstitial fluid that are indicative of other markers, such as lactate levels, ketones, or ionic potassium, which may improve an accuracy in identifying or predicting glucose-based events (e.g., hyperglycemia or hypoglycemia). Additionally or alternatively, the analyte monitoring device 104 may include additional sensors and/or architectures to the analyte sensor 202 to detect those analytes indicative of the other markers.

In another example, the analyte sensor 202 (or an additional sensor of the analyte monitoring device 104—not shown) can include a first and second electrical conductor, and the sensor module 204 can electrically detect changes in electric potential across the first and second electrical conductor of the analyte sensor 202. In this example, the sensor module 204 and the analyte sensor 202 are configured as a thermocouple such that the changes in electric potential correspond to temperature changes. In some examples, the sensor module 204 and the analyte sensor 202 are configured to detect a single analyte, e.g., glucose. In other examples, the sensor module 204 and the analyte sensor 202 are configured to use diverse sensing modes to detect multiple analytes, e.g., ionic sodium, ionic potassium, carbon dioxide, and glucose. Alternatively or additionally, the analyte monitoring device 104 includes multiple sensors to detect not only one or more analytes (e.g., ionic sodium, ionic potassium, carbon dioxide, glucose, and insulin) but also one or more environmental conditions (e.g., temperature, moisture, movement). Thus, the sensor module 204 and the analyte sensor 202 (as well as any additional sensors) may detect the presence of one or more analytes, the absence of one or more analytes, and/or changes in one or more environmental conditions. Further, the sensor module 204 and the analyte sensor 202 (as well as any additional sensors) may be configured to output a signal indicative of an amount (e.g., level) of the one or more analytes in the blood or interstitial fluid. As noted above, the analyte monitoring device 104 may be configured to produce data describing a single analyte (e.g., glucose) or multiple analytes.

In one or more implementations, the sensor module 204 may include a processor and memory (not shown). The sensor module 204, by leveraging the processor, may generate the analyte measurements 110 based on the communications with the analyte sensor 202 that are indicative of the above-discussed changes. Based on the above-noted communications from the analyte sensor 202, the sensor module 204 is further configured to generate communicable packages of data that include at least one analyte measurement 110. In this example 200, the sensor data 118 represents these packages of data. Additionally or alternatively, the sensor module 204 may configure the sensor data 118 to include additional data, including, by way of example, supplemental sensor information 214. The supplemental sensor information 214 may include a sensor identifier, a sensor status, temperatures that correspond to the analyte measurements 110, measurements of other analytes that correspond to the analyte measurements 110, and so forth. It is to be appreciated that the supplemental sensor information 214 may include a variety of data that supplement at least one analyte measurement 110 without departing from the spirit or scope of the described techniques.

In implementations where the analyte monitoring device 104 is configured for wireless transmission, the transmitter 208 may transmit the sensor data 118 as a stream of data to a computing device (e.g., a computing device of the observation analysis platform 108 of FIG. 1). Alternatively or additionally, the sensor module 204 may buffer the analyte measurements 110 and/or the supplemental sensor information 214 (e.g., in memory of the sensor module 204 and/or other physical computer-readable storage media of the analyte monitoring device 104) and cause the transmitter 208 to transmit the buffered sensor data 118 later at various regular or irregular intervals, e.g., time intervals (approximately every second, approximately every thirty seconds, approximately every minute, approximately every five minutes, approximately every hour, and so on), storage intervals (when the buffered analyte measurements 110 and/or supplemental sensor information 214 reach a threshold amount of data or a number of measurements), and so forth. It should be appreciated that in some implementations, the analyte monitoring device 104 can vary in numerous ways from the example described above without departing from the spirit or scope of the described techniques.

With respect to the supplemental sensor information 214, the sensor identifier represents information that uniquely identifies the analyte sensor 202 from other sensors, such as other sensors of other analyte monitoring devices, other sensors implanted previously or subsequently in the skin 206, and so on. By uniquely identifying the analyte sensor 202, the sensor identifier may also be used to identify other aspects about the analyte sensor 202, such as a manufacturing lot of the analyte sensor 202, packaging details of the analyte sensor 202, shipping details of the analyte sensor 202, and so on. In this way, various issues detected for sensors manufactured, packaged, and/or shipped in a similar manner as the analyte sensor 202 may be identified and used in different ways, e.g., to calibrate the analyte measurements 110, to notify users of defective sensors, to notify manufacturing facilities of machining issues, and so forth.

The sensor status of the supplemental sensor information 214 represents a state of the analyte sensor 202 at a given time, e.g., a state of the sensor at a same time one of the analyte measurements 110 is produced. To this end, the sensor status may include an entry for each of the analyte measurements 110, such that there is a one-to-one relationship between the analyte measurements 110 and statuses captured in the supplemental sensor information 214. For example, the sensor status may describe an operational state of the analyte sensor 202. In one or more implementations, the sensor module 204 may identify one of a number of predetermined operational states for a given analyte measurement 110. The identified operational state may be based on the communications from the analyte sensor 202 and/or characteristics of those communications.

By way of example, the sensor module 204 may include (e.g., in memory or other storage) a lookup table having the predetermined number of operational states and bases for selecting one state from another. For instance, the predetermined states may include a “normal” operation state where the basis for selecting this state may be that the communications from the analyte sensor 202 fall within thresholds indicative of normal operation, e.g., within a threshold of an expected time, within a threshold of expected signal strength, an environmental temperature is within a threshold of suitable temperatures to continue operation as expected, and so forth. The predetermined states may also include operational states that indicate one or more characteristics of the analyte sensor 202's communications are outside of normal activity and may result in potential errors in the analyte measurements 110.

For example, bases for these non-normal operational states may include receiving the communications from the analyte sensor 202 outside of a threshold expected time, detecting a signal strength of the analyte sensor 202 outside a threshold of expected signal strength, detecting an environmental temperature outside of suitable temperatures to continue operation as expected, detecting that the person 102 has rolled (e.g., in bed) onto the analyte monitoring device 104, and so forth. The sensor status may indicate a variety of aspects about the analyte sensor 202 and the analyte monitoring device 104 without departing from the spirit or scope of the described techniques.

Having considered an example of an environment and an example of a wearable analyte monitoring device, consider now a discussion of some examples of details of the techniques for health condition prediction using analyte measurements and machine learning in a digital medium environment in accordance with one or more implementations.

Health Condition Prediction

FIG. 3 depicts an example of an implementation 300 in which health-related data, including analyte measurements, are routed to different systems in connection with health condition prediction.

The illustrated example implementation 300 includes components from FIG. 1, including the observation analysis platform 108 and the person 102. As such, components previously introduced in FIG. 1 are numbered the same and will not be re-introduced. As will be elaborated below, the implementation 300 may be used to classify a status of the person 102 with respect to a specific health condition. For example, the status may include an absence of the specific health condition, a presence of the specific health condition, and/or a severity of the specific health condition. In some examples, the status may further include symptoms or other adverse effects of the health condition that the person 102 is predicted to experience, co-morbidities that the person 102 is predicted to experience, and so forth. It may be understood that as used herein, the term “health condition” broadly includes diseases, disorders, and nonpathological conditions that may receive medical treatment. The term “disease” may refer to any health condition that impairs normal functioning of the body. As such, diseases such as diabetes may be referred to as health conditions herein. Further, “a disease” or “a health condition” may include a group of diseases or health conditions that may be sub-classified into different types. For example, diabetes may be sub-classified as Type 1 diabetes, Type 2 diabetes, GDM, cystic fibrosis diabetes, and so on.

The illustrated example implementation 300 also depicts devices 302 associated with the person 102 that may provide the analyte measurements 110 to the observation analysis platform 108 and/or the storage device 112 in connection with health condition prediction. The devices 302 depicted include the analyte monitoring device 104, worn by the person 102 during the observation period to produce the analyte measurements 110, along with additional devices external to the analyte monitoring device 104. Specifically, the additional, external devices depicted include a mobile phone and a smart watch, although various other devices may be configured to provide the analyte measurements 110 to the observation analysis platform 108 and/or the storage device 112 in one or more implementations. Other examples of the devices 302 may include laptops, tablet devices, wearable health trackers, and so on.

As mentioned above, the analyte measurements 110 may be communicated or otherwise provided via wired or wireless connections to the observation analysis platform 108 and/or the storage device 112. For example, the analyte monitoring device 104 may provide the analyte measurements 110 to the observation analysis platform 108 and/or the storage device 112 via a wired or wireless connection as discussed above. In scenarios where one of the additional, external devices 302 provides the analyte measurements 110, the analyte measurements 110 may first be provided from the analyte monitoring device 104 to the additional, external device, such that the additional, external device communicates or otherwise provides the analyte measurements 110 to the observation analysis platform 108 and/or the storage device 112.

In these scenarios, the additional, external devices 302 may act as an intermediary between the analyte monitoring device 104 and the observation analysis platform 108 and the storage device 112, such that the external devices 302 are used to route the analyte measurements 110 from the analyte monitoring device 104 to the observation analysis platform 108 and/or the storage device 112. Alternatively or additionally, other devices may route the analyte measurements 110 from the analyte monitoring device 104 to the observation analysis platform 108 and/or the storage device 112. Those other devices may include dedicated devices that are configured to extract the data from the analyte monitoring device 104 and that are associated with an entity involved in the health condition prediction, such as a health care provider, hospital, pharmacy, telemedicine service, medical testing laboratory, and so on.

The illustrated example implementation 300 also includes a user population 304. The user population 304 represents multiple users that correspond to persons that have worn analyte monitoring devices, such as the analyte monitoring device 104. It follows then that the analyte measurements 110 of these other users are provided by their respective monitoring devices and/or by external computing devices to the observation analysis platform 108 and/or the storage device 112. In one or more implementations, the user population 304 includes users selected as part of one or more studies conducted, at least in part, for the purpose of collecting data (including the analyte measurements 110) so that the data can be used to generate one or more models using machine learning, e.g., using supervised learning, unsupervised learning, reinforcement learning, and so forth that may be used to classify a specific health condition.

Alternatively or in addition, the user population 304 may include users for which a classification for the health condition was previously generated based on his or her analyte measurements produced during an observation period involving the analyte monitoring device 104 in a similar manner as the health condition prediction is generated for the person 102. Data that are produced prior to the health condition prediction for the person 102 and in connection with studies carried out to collect the data are referred to as “historical” data because they are produced at a point in time before the person 102's analyte measurements 110 are produced. Similarly, data produced prior to the health condition prediction of the person 102 and in connection with the health condition predictions of other users are also historical data. In accordance with the described techniques, the historical data include, for example, historical analyte measurements and historical outcome data. These historical data are used along with machine learning to train or otherwise learn an underlying model for health condition prediction and/or classification, as described in more detail in relation to FIG. 5. In contrast to the historical analyte measurements of the user population 304, the analyte measurements 110 for the health condition prediction of the person 102 may be new analyte measurements.

By way of example, studies to collect data in connection with health condition prediction may involve participants wearing an analyte monitoring device over a time period of multiple days to produce the analyte measurements 110 for those participants. The time period may have a same or different duration from the observation period used to produce the person 102's analyte measurements 110 without departing from the spirit or scope of the described techniques. In addition to collecting the analyte measurements 110, such studies may be leveraged to obtain other data about the participants. Outcome data 306 correspond to at least some of these other data and may describe a variety of aspects about users of the user population 304.

In connection with a study for predicting diabetes, for example, participants may, in addition to wearing analyte (e.g., glucose) monitoring devices, be tested using conventional techniques that produce one or more diagnostic measures, such as HbA1c, FPG, and/or 2Hr-PG. Independent diagnostic measures 308 represents data describing outcomes of one or more such tests in relation to the users of the user population 304. For example, the independent diagnostic measures 308 may describe results of HbA1c, FPG, 2Hr-PG (or OGTT as a combination of FPG and 2Hr-PG), and/or random plasma glucose (RPG) in relation to the users of the user population 304. As such, the independent diagnostic measures 308 may represent clinically determined health condition classifications of the user population 304. Given this, the analyte measurements 110 of a study participant may be associated with the respective participant's independent diagnostic measures 308, e.g., by labeling the measurements. As discussed in more detail below, machine learning may, through a training process, learn patterns in the analyte measurements 110 that are indicative of particular values of the independent diagnostic measures 308, such as patterns in the analyte measurements 110 that indicate a respective person's HbA1c is likely 10.0. It may be understood that the independent diagnostic measures 308 may be different than those described above when the study includes predicting diseases and conditions that are not diabetes.

As illustrated, the outcome data 306 also includes observed adverse effects 310 and clinical diagnoses 312. The observed adverse effects 310 represents data describing adverse effects experienced by users of the user population 304. By way of example in connection with predicting diabetes, the observed adverse effects 310 may describe whether a user has or has not experienced any of one or more adverse effects associated with Type 2 diabetes, such as diabetic retinopathy, cataracts, glaucoma, blindness, severe hyper- or hypoglycemia, heart and blood vessel disease, neuropathy, erectile dysfunction, kidney failure or end-stage kidney disease, slow healing, hearing impairment, skin conditions (e.g., bacterial and fungal infections), sleep apnea, and Alzheimer's disease, to name just few.

Additionally or alternatively, the observed adverse effects 310 may describe whether a user has or has not experienced any of one or more adverse effects associated with GDM, such as her baby having excessive birth weight (requiring a C-section birth), an early (preterm) birth, her baby having respiratory distress syndrome, neonatal hypoglycemia, her baby becoming obese or developing Type 2 diabetes later in life, still birth, and so on.

Additionally or alternatively, the observed adverse effects 310 may describe whether a user has or has not experienced one or more adverse effects associated with other types of diabetes, such as effects associated with Type 1 diabetes, cystic fibrosis diabetes, pancreatic diabetes, and so on. It may be understood that the observed adverse effects 310 may be different for predicting diseases and conditions other than diabetes, and diabetes prediction is provided by way of example. Given this, the analyte measurements 110 of a study participant may be associated with the respective participant's observed adverse effects 310, e.g., by labeling the measurements. As discussed in more detail below, machine learning may, through a training process, learn patterns in the analyte measurements 110 that are indicative of occurrence and non-occurrence of the observed adverse effects 310, such as patterns in analyte measurements 110 that indicate a probability of a respective person having a baby with excessive birth weight requiring a C-section.

The clinical diagnoses 312 represents data describing whether users of the user population 304 have been diagnosed (or not) with the health condition by a clinician or whether they have been provisionally or preliminarily diagnosed with the health condition. By way of example, the diagnoses may be made by a clinician based on one or more of the independent diagnostic measures 308 and/or the observed adverse effects 310. As such, the diagnoses may provide clinically verified health condition classifications. Additionally or alternatively, the clinical diagnoses 312 may be configured to represent labeling based on diagnostic tests that are not approved for diagnosis by, for example, the Food and Drug Administration (FDA) or the clinical community at large. In the example of diabetes, the values of the clinical diagnoses 312 may indicate a respective user is clinically diagnosed with diabetes (or some type of diabetes), is clinically diagnosed with prediabetes (or any of the different types of prediabetes), is provisionally or preliminarily diagnosed with diabetes, does not have diabetes (i.e., is screened), is diagnosed with diabetes using a non-approved test, or is diagnosed with prediabetes using a non-approved test, to name just a few. Given this and the independent diagnostic measures 308, for instance, the analyte measurements 110 may be associated with a respective study participant's independent diagnostic measures 308 and the respective participant's clinical diagnoses 312.

The machine learning may, through training, learn patterns in the analyte measurements 110 that are indicative of particular values of the independent diagnostic measures 308 and further are indicative of different clinical diagnoses 312. As an illustrative example, the machine learning may learn patterns in analyte measurements 110 that indicate a person's HbA1c is likely 6.0 (e.g., “estimated A1c”) and further that a clinician's analysis likely results in a diagnosis of prediabetes. Although this example is discussed in relation to the person's HbA1c, it is to be appreciated that a clinical diagnosis of prediabetes or diabetes may be made based on different measurements (e.g., FPG) and/or observations (e.g., weight gain, neuropathy, and sleep apnea) without departing from the spirit or scope of the described techniques.

In one or more implementations, the outcome data 306 may include or may be usable as labels. For example, a value of each independent diagnostic measure 308 may be used to label the analyte measurements 110 of a respective user of the user population 304. Alternatively or in addition, labels indicative of observed adverse effects 310 experienced by the respective user may be used to label the analyte measurements 110 of the respective user. Alternatively or in addition, labels indicative of the clinical diagnoses 312 may be used to label the analyte measurements 110 of the respective user. For example, the analyte measurements 110 of a user clinically diagnosed with prediabetes may be associated with a ‘prediabetes’ label whereas the analyte measurements 110 of a different user clinically diagnosed with diabetes may be associated with a ‘diabetes’ label. Although the independent diagnostic measures 308, the observed adverse effects 310, and the clinical diagnoses 312 are depicted in the example implementation 300, it is to be appreciated that the outcome data 306 may include data describing different, additional, or fewer aspects of users of the user population 304 without departing from the spirit or scope of the described techniques.

As depicted in the illustrated example implementation 300, the analyte measurements 110 and the outcome data 306 of users of the user population 304 is communicated or otherwise provided to the observation analysis platform 108 and/or the storage device 112. In addition to the analyte measurements 110 and the outcome data 306, additional data describing other aspects of users of the user population 304 may be obtained by the observation analysis platform 108 and/or the storage device 112. By way of example, this additional data may include demographic data (e.g., age, gender, ethnicity), medical history data (e.g., height, weight, body mass index (BMI), body fat percentage, presence or absence of various conditions), stress data, nutrition data, exercise data, prescription data, height and weight data, occupation data, and so forth. These types of additional data are merely examples, and the additional data may include more, fewer, or different types of data without departing from the spirit or scope of the techniques described herein. In one or more implementations, the observation analysis platform 108 and/or the storage device 112 may obtain such additional data (or at least some of the additional data) about the person 102 as well as about the users of the user population 304.

Notably, the illustrated example implementation 300 depicts the observation analysis platform 108 and the storage device 112 separately and also depicts a dashed arrow between the storage device 112 and the observation analysis platform 108. Generally speaking, this arrow represents that the data maintained in the storage device 112 may be obtained by the observation analysis platform 108 from the storage device 112. Said another way, the data maintained by the storage device 112 may be provided to the observation analysis platform 108. As discussed above, the storage device 112 may store the analyte measurements 110 of the person 102 as well as the analyte measurements 110 and the outcome data 306 of the user population 304.

In one or more implementations, the observation analysis platform 108 and the storage device 112 may correspond to a same entity, such as a provider of analyte monitoring devices (e.g., the analyte monitoring device 104) and services related to analyte monitoring. In such implementations, the observation analysis platform 108 and the storage device 112 may be implemented in the “cloud,” across multiple computing devices (e.g., servers) and storage resources allocated to or otherwise associated with the entity (e.g., via a subscription or ownership). To this end, the analyte measurements 110 of the person 102 as well as the analyte measurements 110 and the outcome data 306 of the user population 304 may be obtained by the observation analysis platform 108 from the storage device 112 in ways that a server associated with a service provider obtains data from storage associated with that service provider.

In other implementations, the observation analysis platform 108 and the storage device 112 may correspond to different entities. By way of example, the storage device 112 may correspond to a first entity, such as a computing device (e.g., mobile phone or tablet device) of the person 102, and the observation analysis platform 108 may correspond to a second entity, such as a provider of analyte monitoring devices and services related to analyte monitoring. In this example, the observation analysis platform 108 may implemented, at least in part, as an application of the second entity, running on the person 102's computing device. Alternatively or additionally, the observation analysis platform 108 may be implemented using a server device of the second entity. In the application implementation, the second entity's application may obtain one or more of the analyte measurements 110 of the person 102, the analyte measurements 110 of the user population 304, or the outcome data of the user population 304 from the storage device 112 implemented locally on the computing device, e.g., over a bus or other local transmission means of the computing device. In the server implementation, the server of the second entity may obtain data from the storage device 112, implemented on the computing device, over one or more networks, such as the Internet.

In another example where the observation analysis platform 108 and the storage device 112 correspond to different entities, the storage device 112 may correspond to a first entity, such as a provider of the analyte monitoring devices and services related to analyte monitoring (or limited services related to analyte monitoring). In this latter example, the observation analysis platform 108 may correspond to a second, different entity, such as a service provider, e.g., a data partner of the first entity. In this example, the second entity may be considered a “third party” in relation to the entity corresponding to the storage device 112 (and the analyte monitoring device 104). When it corresponds to a data partner, the observation analysis platform 108 may obtain data from the first entity (i.e., the storage device 112) in accordance with one or more legal agreements between the first and second entities. Provision of the data maintained in the storage device 112 to the observation analysis platform 108 may be controlled by an application programming interface (API).

In this type of scenario, such the API may be considered an “egress” for data, such as the analyte measurements 110 and the outcome data 306. By “egress” it is meant that a flow of data is generally outward from the first entity to a third party (e.g., the second entity). In the context of data provision, the API may expose one or more “calls” (e.g., specific formats for data requests) to the third party. By way of example, the API may expose those calls to the third party after the third party enters into an agreement, e.g., with a business corresponding to the first entity, that allows the third party to obtain data from the storage device 112 via the API. As part of this agreement, the third party may agree to exchange payment in order to obtain data from the first entity. Alternatively or additionally, the third party may agree to exchange data that it produces, e.g., via an associated device, in order to obtain data from the first entity. Parties that enter into agreements to obtain data (e.g., the analyte measurements 110) from the first entity via an API may be referred to as “data partners.” In operation, the API allows the third party to make a request for data (e.g., analyte measurements 110 and/or the outcome data 306) maintained in the storage device 112 in a specific request format, and if the request is made in the specific format, then the first entity provides the requested data in a specific response format. The requested data may be provided in the specific response format in one or more communications (e.g., packets) over a network, e.g., the Internet. Examples of a second entity that may be considered a “third party” include various service providers, such as service providers that provide one or more health monitoring/tracking services, fitness related services, telemedicine services, medical testing laboratory services, and so forth. Indeed, the storage device 112 and the observation analysis platform 108 may be implemented using a variety of devices and/or resources (e.g., computing, communication, storage, etc.), and divisions (or not) between the entities corresponding to the various devices and/or resources may differ from those described above without departing from the spirit or scope of the techniques described herein.

Regardless, the observation analysis platform 108 is configured to obtain the analyte measurements 110 of the person 102 as well as the analyte measurements 110 and the outcome data 306 of the user population 304 and to process them in accordance with the described techniques. Using the analyte measurements 110 and the outcome data 306 of the user population 304, for example, the prediction system 114 is configured to generate one or more machine learning models, e.g., regression models, neural networks, and/or reinforcement learning agents. Once one or more such models are generated, the prediction system 114 is configured to use those one or more models to process the analyte measurements 110 of the person 102 to predict the health condition classification 116 for the person 102, as will be further described herein with respect to FIG. 7.

In the illustrated example implementation 300, the prediction system 114 is shown outputting a notification 314. The notification 314 may be based on the health condition classification 116 (not shown in FIG. 3) or include the health condition classification 116. Consider an example in which the health condition classification 116 output by the prediction system 114's one or more machine learning models is a label that indicates the person 102 is predicted to have diabetes, e.g., a ‘1’ (where a ‘0’ indicates no diabetes) or a text label such as ‘diabetes’. In this case, simply providing the health condition classification 116 to the person 102 may be undesirable. When such information is not delivered with pertinent educational material or is not delivered in an appropriate setting and in a personalized manner, provision of such information may affect the person 102 in a variety of negative ways, such as by causing confusion, anger, depression, and so on. Accordingly, the notification 314 may simply be based on the health condition classification 116, such as by notifying the person 102 that the results of the observation period are available and instructing them to schedule an appointment with his or her associated health care provider.

By way of contrast, providing the health condition classification 116 to a health care provider of the person 102 may not be undesirable. Instead, providing the health condition classification 116 to the health care provider may be preferred (in contrast to not providing the classification) so that the health care provider can suitably inform the person 102 and develop a treatment plan for the person 102. In such scenarios, the notification 314 may simply correspond to the health condition classification 116. Alternatively, notifications communicated to health care providers (or others) may be configured as reports that include the health condition classification 116 along with other information, such as traces of the person 102's analyte measurements 110 over the observation period, measures derived from those analyte measurements 110, recommendations for treatment (e.g., learned from historical data of the user population 304), and so forth. Examples of these notifications are discussed in more detail in relation to FIGS. 8 and 9

As mentioned above and discussed in further detail below (e.g., with respect to FIG. 6), the one or more machine learning models of the prediction system 114 may be trained with various features of the analyte measurements 110 from the user population 304 predict the health condition classification 116 for the person 102. However, it may be desirable to select features (or combinations of features) that not only have clinical relevance for classifying the health condition, but also are robust (or insensitive) to manufacturing variabilities of the analyte monitoring device 104 (e.g., of the analyte sensor 202 of the analyte monitoring device 104 depicted in FIG. 2) in order to increase an accuracy of the classification. In contrast, using features of the analyte measurements 110 that are sensitive to manufacturing variabilities, such as sensor bias, to predict the health condition classification 116 may result in misclassification. For example, the misclassification may include a false positive for the health condition, a false negative for the health condition, and the like.

As such, FIG. 4 depicts an example of an implementation 400 for selecting features for training and using the one or more machine learning models for classifying the health condition. Components previously introduced in FIGS. 1-3 are numbered the same and will not be re-introduced.

The implementation 400 includes a model manager 402 that is configured to, among other tasks, evaluate and select features of the analyte measurements 110 that are robust for accurately classifying a targeted health condition, even in the presence of observed variances in the analyte measurements 110 that may be caused by manufacturing variances of the analyte sensor 202 (not shown in FIG. 4). The model manager 402 is depicted including a plurality of modules, including a preprocessing manager 404, a variance simulator 406, a feature constructor 408, a predictor 410, and an evaluator 412. A “module” may include a hardware and/or software system that operates to perform one or more functions, such as the functions that will be described below. For example, a module may include or may be included in a computer processor, a controller, or another logic-based device that performs operations based on instructions stored on a tangible and non-transitory computer readable storage medium, such as a computer memory. Alternatively, a module may include a hard-wired device that performs operations based on hard-wired logic of the device. The various modules shown in the attached figures, including FIG. 4, may represent the hardware that operates based on software or hardwired instructions, the software that directs hardware to perform the operations, or a combination thereof. The hardware may include electronic circuits that include and/or are connected to one or more logic-based devices, such as microprocessors, processors, controllers, and the like, as will be elaborated with respect to FIG. 15. It may be understood the model manager 402 may include more, fewer, or different modules than those illustrated in FIG. 4 without departing from the scope of the present disclosure.

In the illustrated implementation 400, the model manager 402 is shown obtaining the analyte measurements 110, e.g., from the storage device 112, as well as the outcome data 306 of the user population 304. In general, the preprocessing manager 404 is configured to preprocess the analyte measurements 110 to determine a time-ordered sequence of the analyte measurements 110 according to respective timestamps. Due to corruption and communication errors, the analyte measurements 110 obtained by the prediction system 114 may not only be out of time order but may also be missing one or more measurements. For example, there may be gaps in the time-ordered sequence where one or more measurements are expected. In these instances, the preprocessing manager 404 may be further configured to interpolate the missing analyte measurements and incorporate them into the time-ordered sequence. Additionally or alternatively, the preprocessing manager 404 may be configured to filter out portions of the analyte measurements 110 according to pre-determined criteria, such as to remove corrupted or poor signal quality data. Although this functionality is discussed, in one or more implementations, the analyte measurements 110 as obtained by the prediction system 114 may already be in time order (e.g., one or more time-series of the analyte measurements 110), such that ordering those measurements and interpolating missing measurements is not performed by the preprocessing manager 404.

In the illustrated implementation 400, the preprocessing manager 404 is depicted outputting preprocessed data 414, which includes the analyte measurements 110 in sequential time-series format, as discussed above. The preprocessed data 414 is input into the variance simulator 406. The variance simulator 406 is configured to introduce manufacturing-related sensor variability into the preprocessed data 414 by performing variance simulations. For example, the variance simulator 406 may perform multiple variance simulations over a plurality of simulation rounds, each with a different percent of simulated manufacturing-related analyte sensor variability added to the preprocessed data 414 before the data is input into the feature constructor 408. As one example, the variance simulator 406 may apply different performance variabilities and analyte sensor characteristics to the preprocessed data 414 during each of the plurality of simulation rounds. In some examples, the variance simulator 406 may simulate bias with a fixed variance (e.g., standard deviation) applied per trace of the preprocessed data 414. By way of example, a multiplicative variability (e.g., percent variability) may be drawn from a normal distribution with a fixed standard deviation and a mean swept between a first percent variability and a second percent variability for each simulation round and applied to the preprocessed data 414. The second percent variability may be greater than the first percent variability. As a non-limiting example, the first percent variability may be a negative value (e.g., −8%, or a value between −10% and −5%), and the second percent variability may be a positive value (e.g., 11%, or a value between 10% and 15%). As another non-limiting example, the fixed standard deviation may be 8.

The preprocessed data 414, including that without simulated variability (e.g., 0% variability) and that with known amounts of simulated variability, are input into the feature constructor 408. The feature constructor 408 may be configured to generate data (e.g., one or more feature vectors) that can be evaluated in connection with predicting the health condition classification. In the illustrated implementation 400, the feature constructor 408 is depicted outputting extracted analyte features 416 of the preprocessed data 414, such as from each user of the user population 304. The feature constructor 408 may determine the extracted analyte features 416 by processing the preprocessed data 414 according to one or more predetermined algorithms or functions. Each of the different extracted analyte features 416 may correspond to a different algorithm or function with which the feature constructor 408 processes the preprocessed data 414, at least in some examples.

Here, the extracted analyte features 416 include trend-related features 418, time- and day-related features 420, variability and stability features 422, frequency-related features 424, and value-based features 426. It is to be appreciated that the extracted analyte features 416 may vary from the combination illustrated without departing from the spirit or scope of the described techniques. As discussed above, the preprocessed data 414 may be configured as time-series data, such that each measured analyte level is associated with a point in time and sequenced with respect to time. That is, a first analyte measurement obtained at an earlier time is arranged before a second analyte measurement obtained at a later time in the time-series data. This time-series arrangement of the preprocessed data 414 may enable the feature constructor 408 to extract features related to temporal trends and stability of the analyte measurements 110, which may provide candidates for features that are less sensitive to differences in an output of the analyte sensor 202 due to manufacturing variabilities of the analyte sensor 202.

The trend-related features 418 may include features that describe patterns or trends in the time-series data. For example, the trend-related features 418 may include features that describe relations between analyte values (e.g., levels) obtained at different time points (e.g., 5 minutes apart, 10 minutes apart, 15 minutes apart), such as autocorrelation features. The trend-related features 418 may further include rate-of-change features. These rate-of-change features may correspond to differences in the analyte measurements 110 of the given user of the user population 304 over a unit of time. As an example, the preprocessing manager 404 may determine, between at least two measurements, a difference in the measured amount of analyte and a difference in time such that a change in the amount of analyte over some unit of time may be determined. It is to be appreciated that such rates of change may be determined using more than two of the analyte measurements 110 in the preprocessed data 414. In some examples, the rate-of-change features may include statistics regarding the rates of change in the analyte measurements 110, such as a standard deviation of the rate-of-change of the analyte measurements 110.

The time- and day-related features 420 may include features that describe how analyte dynamics differ day-to-day and/or based on the time of day. Examples of the time- and day-related features 420 may include, for instance, mean analyte levels on a particular day, mean analyte levels at a particular time of day, rates of change in the analyte level on a particular day, rates of change in the analyte level between particular times of day, and corresponding statistics (e.g., standard deviation, coefficient of variation, skew, kurtosis, and so forth). For example, the time- and day-related features 420 may include statistics by day features and statistics by time-of-day features. As another example, the time- and day-related features 420 may include differences between means of the analyte measurements 110 for different days (e.g., a mean of daily difference), differences between means of the analyte measurements 110 for different times of day (e.g., between waking hours and sleeping hours), differences between standard deviations of the analyte measurements 110 for different times of day, and so forth.

The variability and stability features 422 may include features that indicate how stable or variable the analyte measurements are. For example, the variability and stability features 422 may include set point frequency features and peaks features. The set point frequency features may measure, for example, how frequently the analyte level is within a pre-defined range of a set point value. The set point value may be a mode of the analyte measurements 110, for example. The peaks features may include statistics representing analyte dynamics during peaks in the analyte measurements 110 (e.g., where the analyte level reaches a local maximum). For example, the peaks features may measure peak width and/or height relative to a stable baseline analyte level of the user. The stable baseline analyte level may be the mode or a mode proxy, for example.

The frequency-related features 424 may include features extracted from frequency-transformed data of the time-series preprocessed data 414. The frequency-related features 424 may indicate dominant frequencies of analyte variability, for example. The frequency-related features 424 may enable additional information to be extracted from the time-domain data, such as different frequencies into which the time-domain data can be decomposed.

The value-based features 426 may include, for example, mean analyte values and/or median analyte values of the analyte measurements 110 for a given user of the user population 304 and corresponding statistical measures (e.g., standard deviation, skew, kurtosis, coefficient of variation, statistical distributions, and so forth). The value-based features 426 may further include inter-quantile range differences and time-based threshold measures. For example, the value-based features 426 may include a time within range measure, which corresponds to an amount of time during the data collection period that the user's analyte measurements 110 are between a first analyte level and a second analyte level that is less than the first analyte level, corresponding to upper and lower limits of a range, respectively. As another example, the preprocessing manager 404 may determine a time outside range measure, which corresponds to an amount of time over the course of the observation period the user's analyte measurements 110 are outside such a range. Additionally or alternatively, the value-based features 426 may include event occurrence-based features, which may indicate occurrences of the user's analyte measurements 110 increasing above the first analyte level (e.g., a hyperglycemia event) and/or decreasing below the second analyte level (e.g., a hypoglycemia event). Additional examples of the value-based features 426 may include values corresponding to a threshold percentile of the analyte (e.g., a statistically significant threshold percentile such as 94^{th} percentile or greater), a 10 to 90 percentile range of the analyte, and amplitude-based features (e.g., mean amplitude of analyte level excursions), to name just a few.

The predictor 410 is configured to provide model predictions 428 for the extracted analyte features 416, alone and in combination. For example, the model predictions 428 may be binary or probability-based outputs of the health condition predictions for each of the extracted analyte features 416 for each amount of manufacturing variability simulated by the variance simulator 406 (or lack thereof, such as when the percent variability is 0%). The predictor 410 may utilize both univariate feature models that include one of the extracted analyte features 416 and multivariate models that combine at least two features of the extracted analyte features 416 (e.g., bivariate models). As a non-limiting example, the predictor 410 may utilize regression models, e.g., linear or logistic regression models, of the extracted analyte features 416 and their various combinations.

The evaluator 412 is configured to receive the model predictions 428 and categorizes the one or more extracted analyte features 416 according to one or more metrics. Here, the evaluator 412 is shown determining a performance metric 430 and a robustness metric 432. The performance metric 430 may provide an indication of how well each of the one or more extracted analyte features 416 classifies the health condition based on the outcome data 306 for a given user of the user population 304 compared to the model predictions 428, and the robustness metric 432 may provide an indication of how insensitive each of the one or more extracted analyte features 416 is to the simulated manufacturing variance, as will be elaborated below.

The performance metric 430 may measure one or both of a true positive rate where the model prediction 428 of the one or more extracted analyte features 416 accurately predicts that the associated user of the user population 304 has the health condition (e.g., sensitivity) and a true negative rate where the model prediction 428 of the one or more extracted analyte features 416 accurately predicts that the associated user of the user population 304 does not have the health condition (e.g., specificity). The higher the performance metric 430, the more sensitive and specific the one or more extracted analyte features 416 is predicted to be for classifying the health condition, for example. As an example, the performance metric 430 may rank the one or more extracted analyte features 416 on a pre-defined scale, such as a scale from 0 to 1 where 0 refers to none (e.g., 0%) of the corresponding model predictions 428 being accurate and 1 refers to all (e.g., 100%) of the corresponding model predictions 428 being accurate.

The robustness metric 432 may measure how much the performance metric 430 changes per percent variability added to the preprocessed data 414 by the variance simulator 406, as averaged across repetitions for each user of the user population 304. For example, the robustness metric 432 may combine variance sensitivity for the true positive rate and the true negative rate. The robustness metric 432 may rank the one or more extracted analyte features 416 on a pre-defined scale, where a highest value indicates no change in the performance metric 430 in response to the simulated variability and the lowest value indicates maximum change in the performance metric 430 in response to the simulated variability. The higher the robustness metric 432, the more insensitive the corresponding one or more extracted analyte features 416 is predicted to be to manufacturing variations of the analyte sensor. In some examples, in addition to or as an alternative to the robustness metric 432, the evaluator 412 may determine a variance sensitivity metric, which may be an inverse of the robustness metric 432. For example, the higher the variance sensitivity metric, the more sensitive (and less robust) the corresponding one or more extracted analyte features 416 is predicted to be to manufacturing variations in the analyte sensor that may affect the analyte measurements 110. In general, highly robust analyte features (e.g., those having a high robustness metric 432 or a low variability sensitivity metric) correspond to extracted analyte features 416 that exhibit little change in the performance metric 430 with various amounts of simulated variance. As non-limiting examples, trend-related features 418 and variability and stability features 422 may produce extracted analyte features 416 having relatively high robustness metrics 432 (and relatively low variance sensitivity metrics).

Based on the robustness metric 432 and the performance metric 430 of the various extracted analyte features 416, the evaluator 412 is configured to select and output a robust analyte feature combination 434. The robust analyte feature combination 434 may balance performance and robustness in order to generate a health condition classification model that accurately predicts the health condition with high sensitivity and specificity that is relatively unaffected by manufacturing variations that may affect analyte sensor output and performance. As will be further described below with respect to FIG. 5, the robust analyte feature combination 434 may include at least two of the extracted analyte features 416. For example, a model built from the at least two extracted analyte features 416 may exhibit a combination of robustness and performance that is greater than a single extracted analyte feature 416. As an example, a first one of the analyte features of the robust analyte feature combination 434 may have a higher performance metric 430 than a second one of the analyte features in the robust analyte feature combination 434, and the second one may have a higher robustness metric 432 than the first one. In another example, the first one of the analyte features may have a higher performance metric 430 and a higher robustness metric 432 than the second one of the analyte features in the robust analyte feature combination 434.

As a non-limiting example where diabetes is the health condition and glucose is the analyte, the value-based features 426, such as mean glucose, may have relatively high performance metrics 430 since elevated glucose is a hallmark of diabetes. However, at least some of the value-based features 426 also may be relatively sensitive to manufacturing variation of the analyte sensor 202. For example, increasing the simulated positive bias, wherein the glucose measurements are shifted higher, may decrease the true negative rate of univariate models of at least some of the value-based features 426 due to the models incorrectly predicting that users without diabetes have diabetes due to the bias-elevated glucose levels. Similarly, increasing the simulated negative bias, wherein the glucose measurements are shifted lower, may decrease the true positive rate of the univariate models of at least some of the value-based features 426 due to the models incorrectly predicting that users that have diabetes do not have diabetes due to the bias-lowered glucose levels. As a result, the univariate models of at least some of the value-based features 426 may have a relatively low robustness metric 432. Thus, in an example, one of the value-based features 426 may be balanced with a feature having a high robustness metric 432, such as one of the trend-related features 418, in the robust analyte feature combination 434.

Although the above examples describe the robust analyte feature combination 434 as including features belonging to different categories (or types), it may be understood that in other examples, the robust analyte feature combination 434 may include features from the same category. For example, each of the features of the robust analyte feature combination 434 may be one of the trend-related features 418. As another example, each of the features of the robust analyte feature combination 434 may be one of the value-based features 426. As such, it should be understood that the above examples are provided for illustration and not limitation.

To select the robust analyte feature combination 434, the evaluator 412 may filter out multivariate model predictions 428 that have robustness metrics 432 that are less than a pre-determined threshold value. The evaluator 412 may select the multivariate model prediction 428 having the highest performance metric 430 of the remaining candidates, and the associated extracted analyte features 416 may be output as the robust analyte feature combination 434.

Referring now to FIG. 5, an example of a set of graphs 500 for categorizing extracted analyte features (e.g., the extracted analyte features 416 of FIG. 4) to identify the robust analyte feature combination 434 is shown. The graphs 500 include a first graph 502 of univariate feature models and a second graph 504 of bivariate feature models, where the vertical axis of each graph represents a variance sensitivity metric, and the horizontal axis of each graph represents a performance metric. The variance sensitivity metric may be the inverse of the robustness metric 432 of FIG. 4, for example, and the performance metric may be the performance metric 430 of FIG. 4. Each datapoint represents the variance sensitivity metric and the performance metric of each univariate (the first graph 502) or bivariate (the second graph 504) model prediction (e.g., the model predictions 428 of FIG. 4) for predicting the health condition classification using the associated extracted analyte feature(s). That is, the first graph 502 shows individual performance metrics and individual variance sensitivity metrics for model predictions using each extracted analyte feature individually, while the second graph 504 shows the performance and variance sensitivity metrics for model predictions that use two of the extracted analyte features in combination.

Referring first to the first graph 502, a key 506 denotes the category of each extracted analyte feature according to a fill pattern of the datapoint. In this example, high performance features 508 are indicated by dark dot shading and have a relatively higher performance metric than other features, e.g., a performance metric of at least 0.97. As explained above with respect to FIG. 4, the performance metric may rank each extracted analyte feature on a scale from 0 to 1, where 0 refers to none (e.g., 0%) of the univariate model predictions for that feature being accurate at predicting the health condition classification and 1 refers to all (e.g., 100%) of the univariate model predictions for that feature being accurate at predicting the health condition classification. In the example depicted in first graph 502, the high performance features 508 all also have relatively high variance sensitivity metrics, indicating that these high performance features 508 are sensitive to the effects of manufacturing variances on analyte measurements in predicting the health condition classification.

Intermediate features 510 are indicated by medium dot shading that is lighter than the dark dot shading of the high performance features 508. In accordance with the described techniques, the intermediate features 510 are those that have both intermediate performance metrics and intermediate variance sensitivity metrics. In the example shown in the first graph 502 of FIG. 5, for instance, the intermediate features 510 have performance metrics between 0.75 and 0.97 and variance sensitivity metrics between 0.3 and 1.2. Different ranges of performance metrics and variance sensitivity may be used to define the intermediate features 510 (and the other types of features) in variations.

In this example, high robustness features 512 are indicated by horizontal line shading and have a relatively lower variance sensitivity metric than other features, e.g., a variance sensitivity metric of less than 0.2 in the present example. As explained above with respect to FIG. 4, highly robust features are identified as experiencing little change in performance when simulated manufacturing-related variance is added to the analyte measurements. The high robustness features 512 shown in the first graph 502 have performance metrics in a range between 0.80 and 0.95.

In this example, low performance features 514 are indicated by white fill (e.g., no shading). Here, the low performance features 514 are defined as the features having performance metrics less than 0.65, indicating that these features may not be reliability accurate for predicting the health condition classification using a machine learning model. In various scenarios, however, the low performance features 514 also have high robustness, as indicated by their low variance sensitivity metrics.

Thus, in some examples, such as the example shown in FIG. 5, it may be beneficial to combine at least two extracted analyte features in order to produce a feature combination with a higher performance metric and a lower variance sensitivity metric than any single extracted analyte feature. Referring now to the second graph 504, a key 516 denotes the category of the analyte feature combinations depicted in each datapoint according to their fills. In the example depicted in second graph 504, high performance feature combinations 518, which comprise two high performance features of the first graph 502, are indicated by dark dot shading; high performance and intermediate feature combinations 520, which comprise one high performance feature and one intermediate feature of the first graph 502, are indicated by vertical line shading; intermediate feature combinations 522, which comprise two intermediate features from the first graph 502, are indicated by medium dot shading; high robustness and intermediate feature combinations 524, which comprise one high robustness feature and one intermediate feature of the first graph 502, are indicated by diagonal line shading; high robustness feature combinations 526, which comprise two high robustness features of the first graph 502, are indicated by horizontal line shading; and low robustness feature combinations 528, which comprise two low performance features of the first graph 502, are indicated by white fill (or no fill).

A robustness threshold 530 shown in second graph 504 may be used (e.g., by the evaluator 412) to filter out feature combinations of the bivariate models for consideration as the robust analyte feature combination 434. In one or more implementations, the robustness threshold 530 is a non-zero, pre-determined variance sensitivity metric value that is calibrated to distinguish feature combinations that are robust to analyte sensor manufacturing variations from feature combinations that are not robust to the analyte sensor manufacturing variations. As such, feature combinations having variance sensitivity metrics below the robustness threshold 530 are considered robust. As a non-limiting example, the robustness threshold 530 may be 0.5, as depicted in FIG. 5. In other examples, the robustness threshold 530 may be set to another value, such as 0.4.

In still other examples, the robustness metric may be used instead of the variance sensitivity metric. In such examples, the robustness threshold 530 may be a non-zero, pre-determined robustness metric value above which the feature combinations are considered robust to analyte sensor manufacturing variations. As such, when the robustness metric is used instead of the variance sensitivity metric, feature combinations that are less than the robustness threshold 530 may be filtered out of consideration for the robust analyte feature combination 434.

In some examples, such as the example shown in FIG. 5, the feature combination with the greatest performance metric that is not filtered out by the robustness threshold 530 (e.g., is less than the robustness threshold 530 when the robustness threshold 530 is a variance sensitivity metric value) is selected as the robust analyte feature combination 434. The robust analyte feature combination 434 is a high robustness and intermediate feature combination 524 in the example depicted in second graph 504, but in other examples, the robust analyte feature combination 434 may be a different category of feature combinations. As such, it may be understood that the examples shown in FIG. 5 are for illustration and not limitation.

In some implementations, additional aspects may be evaluated in selecting the robust analyte feature combination 434 (e.g., in addition to the performance metric and the robustness threshold 530). The additional aspects may include, but are not limited to, an ease of computation of the features in the feature combination, an ability to determine the features in the feature combination using hardware alone, an amount of data needed to compute the features in the feature combination (e.g., collection over multiple days versus collection over a few hours), and so forth. Accordingly, in some implementations, an additional metric (or score) may be determined based on these additional aspects (e.g., via the evaluator 412) for a selected number of top candidate feature combinations having the highest performance metrics that are not filtered out by the robustness threshold 530. As a non-limiting example, the additional metric may be determined for candidate feature combinations having the three greatest performance metrics. In some implementations, the additional metric may be added to the performance metric of each of the selected number of top candidate feature combinations, and the top candidate feature combination having the highest sum may be selected as the robust analyte feature combination 434. In this way, the robust analyte feature combination 434 may include an easier to analyze combination of features than the feature combination with the greatest performance metric.

Further, although FIG. 5 shows using the robustness threshold 530 to filter the feature combinations of the second graph 504, in other examples, a performance metric threshold may be used to filter the feature combinations instead of or in addition to the robustness threshold 530. For example, the feature combination with the lowest variance sensitivity metric (or highest robustness metric) that is greater than the performance metric threshold may be selected as the robust analyte feature combination 434. Additional examples of how the robust analyte feature combination 434 may be selected based on the variance sensitivity metric (or the robustness metric) and the performance metric will be described below with respect to FIG. 11.

The robust analyte feature combination 434 determined via the example implementation 400 of FIG. 4 and illustrated in the second graph 504 of FIG. 5 may be used to train a machine learning model to predict health condition classifications. Referring now to FIG. 6, an example of an implementation 600 of the prediction system 114 in greater detail in which a machine learning model 602 is trained via the model manager 402 is shown. Components previously introduced in FIGS. 1-4 are numbered the same and will not be re-introduced.

In the illustrated implementation 600, the prediction system 114 includes the machine learning model 602 and the model manager 402, which manages a machine learning model 602. In accordance with the described techniques, the machine learning model 602 may represent a single machine learning model or an ensemble of multiple models. The machine learning model 602 may correspond to different types of machine learning models, where the underlying models are learned using different approaches, such as using supervised learning, unsupervised learning, and/or reinforcement learning. By way of example, these models may include regression models (e.g., linear, polynomial, and/or logistic regression models), classifiers, neural networks, and reinforcement learning based models, to name just a few.

The machine learning model 602 may be configured as, or include, other types of models without departing from the spirit or scope of the described techniques. These different machine learning models may be built or trained (or the model otherwise learned), respectively, using different data and different algorithms due, at least in part, to different architectures and/or learning paradigms. Accordingly, it is to be appreciated that the following discussion of the model manager 402's functionality is applicable to a variety of machine learning models. For explanatory purposes, however, the functionality of the model manager 402 in training the machine learning model 602 will be described generally in relation to a statistical model and a neural network.

Broadly speaking, the model manager 402 is configured to manage machine learning models, including the machine learning model 602. This model management includes, for example, building the machine learning model 602, training the machine learning model 602, updating this model, and so forth. Specifically, the model manager 402 is configured to carry out this model management using, at least in part, the wealth of data maintained in the storage device 112. As illustrated, this data includes the analyte measurements 110 and the outcome data 306 of the user population 304. Said another way, the model manager 402 builds the machine learning model 602, trains the machine learning model 602 (or otherwise learns the underlying model), and updates this model using the analyte measurements 110 and the outcome data 306 of the user population 304. For example, the model manager 402 may select the robust analyte feature combination 434 based on the analyte measurements 110 and the outcome data 306 of the user population 304 according to the implementation 400 discussed above with respect to FIG. 4 and use the robust analyte feature combination 434 as input for training the machine learning model 602. In implementations where the machine learning model 602 receives data in addition to the robust analyte feature combination 434 as input, such as additional data from the user population 304, the model manager 402 also uses such additional data to build, train, and update the machine learning model 602.

In one or more implementations, the model manager 402 generates training data to train the machine learning model 602 or to otherwise learn parameters of the model. Broadly speaking, generation of the training data is dependent on the health condition classification the machine learning model is designed to output. This training data will be different, for instance, if the machine learning model 602 is configured to generate predictions of diagnostic measures of a person, adverse effects the person is predicted to experience, or clinical diagnoses of the person. Regardless of the outcome to be predicted, generating the training data may include time sequencing the analyte measurements 110 of the user population 304 (if the analyte measurements 110 are not already time-sequenced) and extracting analyte features from those time-sequenced analyte measurements 110, such as described above with respect to FIG. 4. The model manager 402 may leverage the functionality of the preprocessing manager 404 to form preprocessed data 414 of time sequenced analyte measurements 110 and the feature constructor 408 to extract analyte features, for instance, in a similar manner as discussed above in relation to generating the extracted analyte features 416. As an example, once the robust analyte feature combination 434 is selected, the model manager 402 may use the feature constructor 408 to extract the robust analyte feature combination 434 from the training data. The model manager 402 may not utilize the variance simulator 406 in training the machine learning model 602, at least in some examples.

Generating the training data also includes associating the traces of the analyte measurements 110 or the robust analyte feature combination 434 extracted from the analyte measurements 110 with the outcome data 306 of a respective user of the user population 304. Given this, an analyte trace or an extracted analyte feature corresponding to a particular user is associated with outcome data 306 of the particular user. By way of example, a particular user may have been clinically diagnosed with diabetes and his or her glucose may have been above a threshold for an amount of time corresponding to 27% of an observation period. Given this, the model manager 402 may form a training instance that includes an input portion with a value indicating the user's time above threshold is 27% and having an associated output portion with a value indicating the person has diabetes, e.g., ‘1’ or some other corresponding value.

In one or more implementations, the model manager 402 may build a statistical model by extracting from the outcome data 306 observed values or labels corresponding to at least one type of outcome, such as values of the clinical diagnoses 312, e.g., ‘diabetes’, ‘prediabetes’, ‘no diabetes’, or values indicative of those labels. Once built, the statistical model is configured to predict values or labels of this at least one outcome type and output them as the health condition classification 116; values or labels indicative of the at least one outcome type do not serve as input to the model. In scenarios where the statistical model is a regression model, for instance, outcome values or labels may correspond to one or more dependent variables. In contrast, each feature of the robust analyte feature combination 434 extracted from the analyte measurements 110 may serve as input to the model. Thus, in scenarios where the machine learning model 602 is configured as a statistical model, the at least two analyte features of the robust analyte feature combination 434 may correspond to at least two explanatory (or independent) variables.

Given the set of outcome values or labels from the outcome data 306 and the set of values of the robust analyte feature combination 434 extracted from the analyte measurements 110, the model manager 402 uses one or more known approaches for “fitting” these sets of values to an equation so that it produces the outcome values or labels responsive to input of the extracted analyte feature values, within some tolerance. Examples of such fitting approaches include using a least squares approach, using a least absolute deviations regression, minimizing a penalized version of the least squares cost function (e.g., ridge regression or lasso), and so forth. By “fitting” it is meant that the model manager 402 estimates model parameters for the equation using the one or more approaches and these sets of values of the training data.

The estimated parameters include, for instance, weights to apply to values of the independent variables (e.g., the robust analyte feature combination 434) when they are input to the machine learning model during operation. The model manager 402 incorporates these parameters estimated from fitting the observed values of the user population 304 into the equation to generate the machine learning model 602 as a statistical model. In operation, the prediction system 114 inputs values of the independent variables (e.g., values of the robust analyte feature combination 434) into the statistical model (e.g., as one or more vectors or a matrix), the statistical model applies the estimated weights to these input values, and then outputs values or labels for the one or more dependent variables. This output corresponds to the health condition classification 116.

In the following discussion, the capabilities of the model manager 402 to build and train machine learning models is discussed in relation to a configuration of the machine learning model 602 corresponding to or including at least one neural network.

With respect to the training data used, the model manager 402 may, as noted above, generate instances of the training data including an input portion and an expected output portion, i.e., a ground truth for comparison to the model's output during training. The input portion of a training data instance may correspond to one or more traces of analyte measurements 110 and/or one or more extracted features in the robust analyte feature combination 434 for a particular user. The output portion may correspond to one or more values of the particular user's outcome data 306, e.g., a value indicative of a clinical diagnosis of the health condition or a corroborating clinical diagnostic value (e.g., the user's observed HbA1c when the health condition is diabetes). Again, whether traces are used for training as well as the features of the robust analyte feature combination 434 used for training and which outcome data is used for training depends on the data the machine learning model 602 is designed (and trained) to receive as input and the data it is designed (and trained) to output.

The model manager 402 uses the training input portions along with the respective expected output portions to train the machine learning model 602. In the context of training, the model manager 402 may train the machine learning model 602 by providing an instance of data from the set of training input portions to the machine learning model 602. Responsive to this, the machine learning model 602 generates a prediction of a disease of condition classification, such as by predicting a value indicative of a clinical diagnosis. The model manager 402 obtains this training prediction from the machine learning model 602 as output and compares the training prediction to the expected output portion that corresponds to the training input portion. For example, if the machine learning model 602 outputs a diabetes classification indicating that the user has diabetes, then this prediction is compared to the output data (e.g., which classifies the user as having diabetes or no diabetes) to determine if the prediction was correct. Based on this comparison, the model manager adjusts internal weights of the machine learning model 602 so that the machine learning model can substantially reproduce the expected output portion when the respective training input portion is provided as input in the future.

This process of inputting instances of the training input portions into the machine learning model 602, receiving training predictions from the machine learning model 602, comparing the training predictions to the expected output portions (observed) that correspond to the input instances (e.g., using a loss function such as mean squared error), and adjusting internal weights of the machine learning model 602 based on these comparisons, can be repeated for hundreds, thousands, or even millions of iterations—using an instance of training data per iteration.

The model manager 402 may perform such iterations until the machine learning model 602 is able to generate predictions that consistently and substantially match the expected output portions. The capability of a machine learning model to consistently generate predictions that substantially match expected output portions may be referred to as “convergence.” Given this, it may be said that the model manger 402 trains the machine learning model 602 until it “converges” on a solution, e.g., the internal weights of the model have been suitably adjusted due to training iterations so that the model consistently generates predictions that substantially match the expected output portions.

As noted above, the machine learning model 602 may be configured to receive input in addition to the analyte measurements 110 and/or the robust analyte feature combination 434 extracted from those measurements in one or more implementations. In such implementations, the model manager 402 may form training instances that include the training input portion, the respective expected output portion, and additional input data describing any other aspects of the user population 304 being used to predict health condition classifications, e.g., demographic information, medical history, exercise, and/or stress. This additional data as well as the training input portion may be processed by the model manger 402 according to one or more known techniques to produce an input vector. This input vector, describing the training input portion as well as the other aspects, may then be provided to the machine learning model 602. In response, the machine learning model 602 may generate a prediction of the health condition classification in a similar manner as discussed above, such that the prediction can be compared to the expected output portion of the training instance and weights of the model adjusted based on the comparison.

It may be understood that in some examples, at least portions of the implementation 400 of FIG. 4 and the implementation 600 of FIG. 6 may be performed in combination. For example, the variance simulator 406 may introduce manufacturing-related sensor variability when tuning the machine learning model 602. By way of example, the simulated manufacturing-related sensor variability may be applied to the validation fold of a candidate model of the machine learning model 602, inside of cross validation and before evaluating the candidate model on the validation fold.

Once the machine learning model 602 is trained, it is used to predict health condition classification. In the context of predicting the health condition classification 116 for the person 102 from the person 102's analyte measurements 110, consider the following discussion of FIG. 7.

FIG. 7 depicts an example of an implementation 700 of the prediction system of FIG. 1 in greater detail in which a health condition classification is predicted using machine learning. As such, components previously introduced in FIGS. 1-6 are numbered the same and will not be reintroduced.

In the illustrated implementation 700, the prediction system 114 is shown obtaining the analyte measurements 110, e.g., from the storage device 112 (not shown in FIG. 7). Here, the analyte measurements 110 correspond to the person 102. In the implementation 700, the prediction system 114 is depicted including the preprocessing manager 404, the feature constructor 408, and the machine learning model 602, which are configured to generate a prediction of the health condition classification 116 based on the analyte measurements 110 of the person 102. Although the prediction system 114 is depicted including these three components, it is to be appreciated that the prediction system 114 may have more, fewer, and/or different components to generate the health condition classification 116 based on the analyte measurements 110 without departing from the spirit or scope of the described techniques.

In one or more implementations, the analyte measurements 110 are configured as time-sequenced data, such that each of the analyte measurements 110 corresponds to a timestamp, as described above with respect to FIG. 4. As such, the preprocessing manager 404 may be configured to determine a time-ordered sequence of the analyte measurements 110 of the person 102 according to respective timestamps and generate the preprocessed data 414. The preprocessed data 414 may be input into the feature constructor 408, which may be configured to extract analyte features from the preprocessed data 414 (e.g., feature vectors) that can be provided as input to the machine learning model 602 and data that can be reported in connection with the health condition classification 116 (e.g., included as part of the notification 314 shown in FIG. 3). In particular, the machine learning model 602 may be trained to predict the condition classification 116 using the robust analyte feature combination 434 selected via the implementation 400 of FIG. 4, such as described above with respect to implementation 600 of FIG. 6. As such, in the illustrated implementation 700, the feature constructor 408 is depicted outputting the robust analyte feature combination 434. The feature constructor 408 may determine the robust analyte feature combination 434 by further processing the preprocessed data 414 of the analyte measurements 110 according to one or more predetermined algorithms or functions. Each of the different extracted analyte features of the robust analyte feature combination 434 may correspond to a different algorithm or function with which the feature constructor 408 processes the preprocessed data 414 of the analyte measurements 110.

In addition to the robust analyte feature combination 434, the feature constructor 408 may also incorporate features from additional data that describe different aspects of the person 102, at least in some examples. This additional data may include data of a second, different analyte than the analyte measurements 110 (e.g., lactate measurements when the analyte measurements 110 are glucose measurements), environmental data (e.g., the person's temperature), already-observed adverse effects data (e.g., data describing that any of a variety of adverse effects associated with the health condition classification that have already been observed), demographic data (e.g., describing age, gender, ethnicity) collected through a questionnaire or otherwise obtained, medical history data, stress data, nutrition data, exercise data, prescription data, height and weight data, occupation data, and so forth. In other words, the data provided as input to the machine learning model 602 or an ensemble of the machine learning models may, in one or more implementations, describe a variety of aspects about the person 102 (e.g., as features of input feature vectors) in addition to the robust analyte feature combination 434 without departing from the spirit or scope of the described techniques. In such scenarios, the machine learning model 602 is trained using similar historical data of the user population 304 (see FIG. 6).

Although the illustrated implementation 700 depicts the feature constructor 408 processing the preprocessed data 414 to produce the robust analyte feature combination 434 and using those features as input to the machine learning model 602 (e.g., feature vectors indicative of extracted features), in one or more implementations, the feature constructor 408 may also generate feature vectors that represent (alone or with other features) one or more time-series of the analyte measurements 110 (e.g., traces). Thus, the input data to the machine learning model 602 may correspond to, or otherwise include, a vectorized time-series of the analyte measurements 110 or multiple vectorized time-series of the analyte measurements 110. In implementations where the time-series of the analyte measurements 110 are vectorized, the machine learning model 602 may, for example, correspond to a neural network. In implementations where the robust analyte feature combination 434 are vectorized, the machine learning model may, for example, correspond to a multivariate regression model, e.g., a multivariate linear or logistic regression model.

Responsive to receiving the input data from the feature constructor 408, the machine learning model 602 is configured to generate and output the health condition classification 116. Specifically, the machine learning model 602 may be trained to output the health condition classification 116. By way of example, and as elaborated above with respect to FIG. 6, the machine learning model 602 may be trained, or an underlying representation may be learned, based on one or more training approaches and using historical analyte data and outcome data from which health condition classifications can be derived, such as using the analyte measurements 110 and the outcome data 306 of the user population 304. In accordance with the described techniques, the machine learning model 602 may represent one or more models, including, for instance, a model trained to predict whether the person has the health condition and, in one or more implementations, an additional model to predict whether the person does not have the health condition (e.g., a diabetes classification that can be used to screen the person as not having diabetes with a degree of certainty). Each of the models of a multi-model configuration may receive differently configured input data that describes different aspects, e.g., feature vectors with features that represent different aspects related to the health condition. It is to be appreciated that in other implementations, a single model may be configured to generate both types of predictions. In one or more implementations, the machine learning model 602 may be configured as an ensemble of models that each generates a different prediction related to the health condition than the other models.

The health condition classification 116 may classify the person 102 in terms of one or more outcomes, which correspond to the outcomes described by the outcome data 306 used to train the machine learning model 602. In implementations where the machine learning model 602 is trained, or the model is learned, using the clinical diagnoses 312 of the user population 304, then the machine learning model 602 may classify the person 102's analyte measurements 110 into a class corresponding to one of the diagnoses. In an example implementation where the health condition is diabetes, the machine learning model 602 may classify the analyte measurements 110 of the person 102 as diabetes, prediabetes, or no diabetes. To this end, a health care provider may use the health condition classification 116 to treat the person 102 or develop a treatment plan similarly to how the health care provider would do so if the person 102 were diagnosed with diabetes according to conventional techniques, e.g., HbA1c, FPG, and/or 2Hr-PG.

Similarly, where the machine learning model 602 is trained, or the model is learned, using the observed adverse effects 310 of the user population, then the machine learning model 602 may output probabilities that the person 102's analyte measurements 110 are indicative of the person experiencing the different adverse effects, e.g., a probability from zero to one that the person will experience any of the variety of adverse effects associated with the health condition. In some implementations, there may be a machine learning model trained or built for each effect, such that the machine learning model 602 represents an ensemble of models capable of generating predictions regarding whether the person 102 will or will not experience each effect (or a probability of the person experiencing each effect).

In implementations where the machine learning model is trained, or the model is learned, using the independent diagnostic measures 308 of the user population, then the machine learning model 602 may output a prediction of a value of a particular diagnostic measure. Continuing with the example implementation where the health condition is diabetes, the machine learning model 602 may output a prediction of an HbA1c value, a FPG value, a 2Hr-PG value, or an OGTT value. The health condition classification 116 that is output depends largely on how the machine learning model 602 is trained and the information, specifically, that the health condition classification 116 represents depends on the training. For example, the condition classification 116 may represent a label indicative of whether or not the person has the health condition or is at risk for developing the health condition (e.g., a diabetes label, a prediabetes label, or a no-diabetes label), a label indicative of whether or not the person has a particular type of the health condition (e.g., Type 1 diabetes, Type 2 diabetes, and GDM), a probability, or a measure value. Additionally, different types of machine learning models may be better suited to generate predictions in relation to different types of outcomes that can be represented by the health condition classification 116.

Additionally, the condition classification 116 output by the machine learning model 602 may serve as a basis for a variety of information provided to the person 102 in relation to which the prediction is generated as well as others associated with the person, such as the person 102's health care provider, a caregiver, a telemedicine or health tracking service, and so forth. In the context of information that may be output based on the predictions, consider the following discussion of FIGS. 8-9.

FIG. 8 depicts an example of an implementation 800 of a user interface displayed for notifying a user about a health condition prediction generated based on analyte measurements produced during an observation period. In the following description, reference will be made to components and features illustrated in FIGS. 1-7 for generating the health condition prediction. However, it may be understood that the implementation 800 may notify the user about a health condition prediction generation using other features and components without departing from the scope of the present disclosure.

The illustrated example implementation 800 includes a computing device 802 displaying a user interface 804. In this example implementation 800, the user interface 804 may correspond to the notification 314 of FIG. 3. This example implementation 800 represents a scenario where the notification 314 (e.g., output via the user interface 804) is generated based on the health condition classification 116 introduced in FIG. 1 but does not include the health condition classification 116. Here, the computing device 802 may be associated with the person 102 whose analyte measurements are collected during the observation period and in relation to which the health condition classification 116 is generated. Alternatively, the computing device 802 may be associated with another person affiliated with the person 102, such as a caregiver.

To this end, the user interface 804 may be displayed to notify the person 102 (or the person affiliated with the person 102) about the health condition classification 116 without revealing the predicted classification. This is because output of the health condition classification 116 to the person 102 may affect the person 102 in a variety of negative ways, such as by causing confusion, anger, depression, and so on. In this example implementation 800, the user interface 804 includes a summary about the processing of the person 102's analyte measurements. The user interface 804 also includes a recommendation of actionable behavior based on the health condition classification—in this case recommending that the person 102 follow up with his or her health care provider. In addition, the user interface 804 includes graphical user interface elements 806 that are selectable to carry out the recommended actionable behavior. Each of the graphical user interface elements 806 may be selectable to cause a follow up appointment with a health care provider of the person 102 to be scheduled, such as an appointment at a physical location of the health care provider or an appointment via a telephone or video conference, e.g., in connection with remote health care and/or a telemedicine service. It is to be appreciated that notifications generated based on the health condition classification 116, but that do not include the classification, may be configured in different ways without departing from the spirit or scope of the described techniques.

FIG. 9 depicts an example of an implementation 900 of a user interface displayed for reporting a health condition prediction of a user along with other information produced in connection with the health condition prediction. In the following description, reference will be made to components and features illustrated in FIGS. 1-7 for generating the health condition prediction. However, it may be understood that the implementation 900 may report the health condition prediction based on data generated and analyzed using other features and components without departing from the scope of the present disclosure. The example implementation 900 includes diabetes as the health condition as determined based on glucose measurements obtained from the user. However, it may be understood that the implementation 900 may be adapted for reporting predictions and associated information for other diseases and conditions without departing from the scope of the present disclosure.

The illustrated example implementation 900 includes a display device 902 displaying a user interface 904, which is configured as a report. In this example, the user interface 904 may correspond to the notification 314 of FIG. 3. In contrast to the example implementation 800 depicted in FIG. 8, this example implementation 900 represents a scenario where the notification includes the health condition classification 116 introduced in FIG. 1. In this illustrated example implementation 900, a graphical diagnosis element 906 represents or otherwise indicates the health condition classification 116. Here, the display device 902 may be associated with a health care provider affiliated with the person 102 whose analyte measurements are collected during the observation period and for which the health condition classification 116 is generated.

To this end, the user interface 904 may be displayed to report the health condition classification 116 to the health care provider and to report additional information that may be pertinent to the classification. In operation, the health care provider may independently analyze the additional information reported and provide a different diagnosis from the one indicated by the health condition classification 116. In this example implementation 900, the additional information includes glucose traces 908, 910. The glucose traces 908, 910 represent analyte measurements 110 of the person 102 collected over two days of the observation period. The user interface 904 is also depicted with controls that may allow a user to navigate to other analyte measurements 110 collected, such as traces corresponding to previous or subsequent days of the observation period.

The user interface 904 also includes graphical glucose feature elements 912, which represent or otherwise indicate one or more of the extracted analyte features 416 determined by the feature constructor 408 based on the person 102's analyte measurements 110. In addition to a predicted clinical diagnosis, as indicated by the graphical diagnosis element 906, the user interface 904 also includes predicted adverse effects elements 914 and probability elements 916. The inclusion of the predicted adverse effects elements 914 and the probability elements 916 indicates that the machine learning model 602 introduced in FIG. 6 may be configured (e.g., via configuration as an ensemble of models and/or based on architecture and training) to generate predictions of more than one type of diabetes classification. By way of example, the machine learning model 602 may be configured to predict a clinical diagnosis of the person 102, values for one or more of a plurality of clinical diagnostic values associated with diabetes (e.g., HbA1c, FPG, 2Hr-PG, and OGTT), and probabilities that the person 102 will experience one or more of a plurality of adverse effects of diabetes.

In particular, the predicted adverse effects elements 914 correspond to adverse effects the diabetes classification indicates the person 102 is more likely to experience than not, e.g., based on a probability output by the machine learning model of experiencing those effects being greater than 50%. It is to be appreciated that adverse effects for which the machine learning model 602 predicts have any probability of occurring may also be output in one or more scenarios along with the corresponding probabilities. The probability elements 916 include the probabilities that the adverse effects indicated by the elements 914 will occur. The probabilities indicated by these probability elements 916 may be output by the machine learning model 602 in one or more implementations. It is to be appreciated that reports including the health condition classification 116 may be configured in different ways without departing from the spirit or scope of the described techniques, such as a document suitable for printing.

FIG. 10 depicts an example of an implementation 1000 of a user interface displayed for collecting additional data that can be used as input to machine learning models for generating a health condition prediction. In the following description, reference will be made to components and features illustrated in FIGS. 1-7 and described above for generating the health condition prediction. However, it may be understood that the implementation 1000 may collect additional data for use by other features and components without departing from the scope of the present disclosure.

The illustrated example implementation 1000 includes a computing device 1002 displaying a user interface 1004. In this example implementation 1000, the user interface 1004 may be displayed to collect data about the person 102 in addition to the analyte measurements 110 collected during the observation period. These additional data, along with traces of the analyte measurements 110 and/or one or more of the extracted analyte features 416, may be provided to the machine learning model 602 as input. That is, the additional data may be represented in features of a feature vector input to the model. To train the machine learning model 602, these additional data may also be collected from the users of the user population 304. Accordingly, the user interface 1004 may be displayed to the users of the user population 304 to collect these additional data from those users. The additional data may include, for example, data describing demographics, medical history, exercise, and/or stress.

In the illustrated example implementation 1000, the user interface 1004 includes a variety of graphical elements 1006 with which a user may interact (e.g., select or enter values) to provide additional data about himself or herself. However, it is to be appreciated that the included graphical elements 1006 are merely examples, and a user interface to collect such additional data may be configured in different ways to include more, fewer, or different elements that enable collection of various additional data without departing from the spirit or scope of the described techniques.

Having discussed examples of details of the techniques for health condition prediction using analyte measurements and machine learning, consider now some examples of procedures to illustrate additional aspects of the techniques.

Examples of Procedures

This section describes examples of procedures (e.g., methods) for health condition prediction using analyte measurements and machine learning. Aspects of the procedures may be implemented in hardware, firmware, software, or a combination thereof. The procedures are shown as a set of blocks that specify operations performed by one or more devices and are not necessarily limited to the orders shown for performing the operations by the respective blocks. In some examples, multithreading or parallel processing may be used in executing the described procedures. In at least some implementations the procedures are performed by a prediction system, such as the prediction system 114 of FIG. 1 that makes use of the model manager 402 of FIG. 4. As such, reference will be made to features and components described above with respect to FIGS. 1-6, although it may be understood that the procedures may be performed using similar or different components without departing from the scope of the present disclosure.

FIG. 11 depicts a procedure 1100 in an example of an implementation in which robust analyte feature combinations for predicting a health condition classification are selected based on historical analyte measurements and outcome data of a user population.

Analyte measurements collected by wearable analyte monitoring devices worn by users of a user population are obtained (block 1102). By way of example, the model manager 402 obtains the analyte measurements 110 of users of the user population 304. In some examples, the model manager 402 generates analyte data from the analyte measurements 110, such as via a preprocessing manager 404 of the model manager 402. The analyte data may be preprocessed data (e.g., the preprocessed data 414 of FIG. 4) that may comprise time-ordered and/or filtered sequences of the analyte measurements 110.

Outcome data is obtained that describes one or more aspects of users of the user population that relate to health condition (block 1104). By way of example, the model manager 402 obtains the outcome data 306. In the examples discussed above, the outcome data describes example aspects, such as one or more of the independent diagnostic measures 308 of the users of the user population 304, the observed adverse effects 310 of the users of the user population 304, and the clinical diagnoses 312 of the users of the user population 304.

Analyte sensor manufacturing-related variability is simulated in the analyte data (block 1106). By way of example, the variance simulator 406 of the model manager 402 introduces the analyte sensor manufacturing-related variability into the analyte data generated from the analyte measurements 110. The analyte sensor manufacturing-related variability may refer to differences in responses of different analyte sensors (e.g., the analyte sensor 202 of FIG. 2) to the same analyte levels due to, for example, sensor bias, differing sensor models, differing sensor lots, differing sensor manufacturers, and the like. For example, the variance simulator 406 may perform multiple simulations on each portion of the analyte data (e.g., each trace), and each simulation may introduce a different percentage of simulated manufacturing-related variability to the analyte data. For example, the variance simulator 406 may simulate manufacturing-related variability by applying, to each portion of the analyte data, a multiplicative or additive variance. As one example, the variance is drawn from a normal distribution with a fixed standard deviation and a mean swept within a pre-determined range of manufacturing-related variability percentages. As non-limiting examples, the standard deviation may be set to 8, and the pre-determined range of manufacturing-related variability percentages may be from −8% to 11%. As such, a new manufacturing-related variability value may be determined for each portion of the analyte data during each round of the simulation. As a non-limiting example, the simulation may be performed for 20 to 40 rounds (e.g., 30 rounds).

A plurality of features is extracted from the analyte data (block 1108). By way of example, the feature constructor 408 of the model manager 402 may receive the analyte data, including analyte data without the simulated analyte sensor manufacturing-related variability and analyte data with varying amounts of the simulated analyte sensor manufacturing-related variability from each round of the simulation, and output the extracted analyte features 416. As such, the extracted analyte features 416 may be output for analyte data with each percentage of the simulated manufacturing-related variability, including 0%.

Combinations of the plurality of features are evaluated for a robustness metric associated with a sensitivity to the analyte sensor manufacturing-related variability and a performance metric associated with predicting the health condition classification (block 1110). By way of example, at least two features of the plurality of features are combined as a multivariate model (e.g., a bivariate model), and model predictions of the health condition classification are generated and compared with the outcome data 306. Alternatively, a single feature of the plurality of features may be used in a univariate model. As described with respect to FIG. 4, the performance metric (e.g., the performance metric 430) may rank each candidate combination of the plurality of features according to an accuracy of predicting the disease of condition classification, and the robustness metric (e.g., the robustness metric 432) may be used to rank each candidate combination of the plurality of features according to a percent change of the performance metric per percent change in the simulated manufacturing-related variability. A higher performance metric may indicate that the candidate combination is more sensitive and specific for predicting the disease of condition, and a higher robustness metric may indicate that the performance of the candidate combination is less influenced by the analyte sensor manufacturing-related variability. Alternatively, in place of the robustness metric, a variance sensitivity metric that is the inverse of the robustness metric may be used. A higher variance sensitivity metric may indicate that the performance of the candidate combination is more influenced by the analyte sensor manufacturing-related variability (e.g., is more sensitive to the manufacturing-related variability).

A combination of the plurality of features is selected to be a robust feature combination for predicting the health condition classification via a machine learning model based on the robustness metric and the performance metric (block 1112). As one example, a robustness threshold (e.g., the robustness threshold 530 of FIG. 5) may be used to filter the candidate combinations of the plurality of features such that candidate combinations having robustness metrics below the robustness threshold (or sensitivity metrics above the robustness threshold) are excluded from further consideration. In some examples, the filtered candidate combination that has the highest performance metric may be selected as the robust feature combination (e.g., robust analyte feature combination 434 of FIG. 4). As such, the robust feature combination may be the candidate combination that has the greatest performance metric of those that have robustness metrics greater than the robustness threshold (or variance sensitivity metrics that are less than the robustness threshold).

As another example, a performance metric threshold may be used instead of or in addition to the robustness threshold. For example, the performance metric threshold may be used to filter out candidate combinations of the plurality of features that have performance metrics that are less than the performance metric threshold. The remaining candidate combination that has the highest robustness metric (or lowest variance sensitivity metric) may be selected as the robust feature combination.

As still another example, additional aspects may be evaluated in selecting the robust feature combination. As mentioned above with respect to FIG. 5, these additional aspects may include, but are not limited to, an ease of computation of the features in the candidate feature combination, an ability to determine the features in the candidate feature combination using hardware alone, an amount of data needed to compute the features in the candidate feature combination (e.g., collection over multiple days versus collection over a few hours), and so forth. Accordingly, in some implementations, an additional metric may be determined for a selected number of top candidate feature combinations (e.g., those having the top performance metrics that are not filtered out by the robustness threshold or having the top robustness metrics that are not filtered out by the performance metric threshold). In some implementations, the additional metric may be added with the performance metric and/or robustness metric of each of the top candidate feature combinations (e.g., as a weighted or non-weighted sum), and the top candidate feature combination having the highest total may be selected as the robust feature combination.

As yet another example, the robust feature combination may be selected based on a sum of the robustness metric and the performance metric for each candidate combination in order to maximize a combination of the robustness metric and the performance metric. For example, the candidate combination that has the highest sum of the robustness metric and the performance metric may be selected as the robust feature combination. In some examples, the sum may be a weighted sum in order to give more or less weight to the performance metric relative to the robustness metric.

In this way, a robust analyte feature combination may be selected that may produce a machine learning model with a consistent performance and high accuracy for ranges or expected variations in the analyte measurements 110 due to sensor-to-sensor manufacturing variations and subject-to-subject variations. By using the robust analyte feature combination, a performance and robustness of the resulting machine learning model may be more effectively balanced than can be achieved with a single analyte feature.

FIG. 12 depicts a procedure 1200 in an example of an implementation in which a machine learning model is trained to predict a health condition classification based on robust analyte features extracted from historical analyte measurements and outcome data of a user population.

Analyte measurements collected by wearable analyte monitoring devices worn by users of a user population are obtained (block 1202). By way of example, the model manager 402 obtains the analyte measurements 110 of users of the user population 304. Outcome data is obtained that describes one or more aspects of users of the user population that relate to a health condition (block 1204). By way of example, the model manager 402 obtains the outcome data 306. In the examples discussed above, the outcome data describes example aspects, such as one or more of the independent diagnostic measures 308 of the users of the user population 304, the observed adverse effects 310 of the users of the user population 304, and the clinical diagnoses 312 of the users of the user population 304.

Instances of training data are generated that include a training input portion and an expected output portion, the training input portion including a combination of features of the users' analyte measurements selected based on a robustness metric and a performance metric, and the expected output portion including one or more values of outcome data corresponding to the users (block 1206). In accordance with the principles discussed herein, the combination of features includes at least two extracted features of each user's analyte measurements (e.g., the robust analyte feature combination 434 introduced with respect to FIG. 4). Selecting the combination of features based on the robustness metric and the performance metric is further described above with respect to procedure 1100 of FIG. 11. Further, the expected output portion includes one or more values of the outcome data that corresponds to each user. By way of example, the model manager 402 generates, for each user of the user population 304, instances of training data by associating the combination of features extracted from the user's analyte measurements obtained at block 1202 with the one or more values of the user's outcome data obtained at block 1204. In one or more implementations, the model manager 402 “labels” the combination of features with one or more labels representative of the values of the outcome data corresponding to the user. The outcome data may include clinically verified indications of the health condition classification, for example.

Here, blocks 1208-1214 may be repeated until a machine learning model is suitably trained, such as until the machine learning model “converges” on a solution, e.g., the internal weights of the model have been suitably adjusted due to training iterations so that the model consistently generates predictions that substantially match the expected output portions. Alternatively or in addition, the blocks 1208-1214 may be repeated for a number of instances (e.g., all instances) of the training data.

The training input portion of an instance of training data is provided as input to the machine learning model (block 1208). By way of example, the model manager 402 provides a training input portion of an instance of training data generated at block 1206 as input to the machine learning model 602.

A prediction of a health condition classification is received as output from the machine learning model (block 1210). In accordance with the principles discussed herein, the prediction of the health condition classification corresponds to a same aspect related to health condition as the one or more values of the user's outcome data included in the training instance. By way of example, the machine learning model 602 predicts a health condition classification (e.g., a classification of the user in a ‘the health condition is present’ class, an ‘at risk of developing the health condition’ class, a ‘the health condition is not present’ class, or a value indicative of one of those classes) based on the training input portion provided at block 1208, and the model manager 402 receives the health condition classification as output of the machine learning model 602.

The prediction of the health condition classification is compared to the expected output portion of the instance of training data (block 1212). By way of example, the model manager 402 compares the health condition classification predicted at block 1210 to the expected output portion of the training instance generated at block 1206, e.g., by using a loss function such as mean squared error (MSE). It is to be appreciated that the model manager 402 may use other loss functions during training to compare the predictions of the machine learning model 602 to the expected output without departing from the spirit or scope of the described techniques.

Weights of the machine learning model are adjusted based on the comparison (block 1214). By way of example, the model manager 402 may adjust internal weights of the machine learning model 602 based on the comparing. In one or more implementations, the model manager 402 may optionally leverage one or more hyperparameter optimization techniques during training to tune hyperparameters of the learning algorithm employed.

FIG. 13 depicts a procedure 1300 in an example of an implementation in which a machine learning model predicts a health condition classification based on analyte measurements of a user collected by a wearable analyte monitoring device during an observation period. The machine learning model may be trained according to the procedure 1200, for example.

Analyte measurements of a user are obtained, where the analyte measurements are collected by a wearable analyte monitoring device worn by the user during an observation period (block 1302). By way of example, the machine learning model 602 obtains analyte measurements 110 that are collected by the analyte monitoring device 104 worn by the person 102 (e.g., the user) during an observation period. The analyte monitoring device 104 may be provided as part of an observation kit, for instance, for the purpose of monitoring the person 102's analyte. Regardless of how the analyte monitoring device 104 is obtained by the person 102, the device is configured to monitor analyte levels of the person 102 during an observation period, which lasts for a time period generally spanning multiple days. The analyte monitoring device 104 may be configured with the analyte sensor 202, for instance, which may be inserted subcutaneously into skin of the person 102 and used to measure the analyte in the person 102's blood or interstitial fluid.

Although discussed throughout as being inserted subcutaneously into the person 102's skin, in one or more implementations, the analyte sensor 202 may not be inserted subcutaneously. In such implementations, the analyte sensor 202 may instead be disposed on the person 102's skin or muscle. For example, the analyte sensor 202 may be a patch that adheres to the person 102's skin for the observation period. This patch may then be peeled off. Alternatively or additionally, a non-invasive analyte sensor may be optical based, e.g., using photoplethysmography (PPG). The analyte sensor 202 may be configured in a variety of ways to obtain measurements indicative of the person 102's analyte levels without departing from the spirit or scope of the described techniques.

A robust feature combination is extracted from the analyte measurements, where the robust feature combination is insensitive to manufacturing variability of the analyte sensor 202 and accurate for predicting the health condition classification (block 1304). As described above with respect to FIGS. 4-5 and 11, the robust feature combination may comprise at least two extracted analyte features from a plurality of extracted analyte features 416. By way of example, a first analyte feature of the robust feature combination may have a higher performance metric (e.g., the performance metric 430 of FIG. 4) for accurately predicting the health condition classification and a lower robustness metric, (e.g., the robustness metric 432 of FIG. 4) for insensitivity to manufacturing variability of the analyte sensor, such as analyte sensor bias, and a second analyte feature of the robust feature combination may have a higher robustness metric and a lower performance metric (e.g., than the first analyte feature). As an illustrative example, the first analyte feature may represent the analyte level or degree of analyte elevation in the person 102's blood, such as one of the value-based features 426 of FIG. 4. As another illustrative example, the second analyte feature may represent analyte patterns or trends, such as one of the trend-related features 418 or variability and stability features 422 of FIG. 4.

A health condition classification of the user is predicted by processing the extracted robust feature combination using one or more machine learning models (block 1306). In accordance with the principles discussed herein, the one or more machine learning models are generated based on historical analyte measurements and historical outcome data of a user population, such as according to the procedure 1200 described above with respect to FIG. 12. By way of example, the machine learning model 602 predicts the health condition classification 116. The machine learning model 602 generates this prediction by processing the extracted robust feature combination based on patterns, learned during training, of the robust feature combination and the outcome data 306 of the user population 304. As noted above, the user population 304 includes users that wear wearable analyte monitoring devices, such as the analyte monitoring device 104. By processing the robust feature combination together rather than individual extracted analyte features, performance and manufacturing variation robustness may be balanced for consistent and accurate model performance.

The health condition classification is output (block 1308). By way of example, the machine learning model 602 outputs the health condition classification 116. As discussed throughout, the health condition classification 116 may indicate whether it is predicted the person has health condition or is predicted to experience adverse effects associated with health condition. The health condition classification 116 may also be used to generate one or more notifications or user interfaces based on the classification, such as a report directed to a health care provider that includes the health condition classification (e.g., that the person is predicted to have health condition) or a notification directed to the person 102 that instructs the person to contact his or her health care provider.

Implementation Example: Using a Factory-Calibrated CGM System to Diagnose Type 2 Diabetes

Type 2 diabetes (T2D) is a progressive disease impacting over 400 million people worldwide that can lead to long-term vascular complications. Before progressing to T2D, many first develop prediabetes, which is characterized by mildly elevated glucose levels. For those with prediabetes and T2D, proper diagnosis and management can improve glycemic control, reducing the risk of diabetes-related complications.

As previously mentioned herein, clinical and regulatory communities have developed criteria for diagnosing T2D that are typically based on fasting plasma glucose (FPG), 2-hour plasma glucose (2Hr-PG), random plasma glucose (RPG) values, and hemoglobin HbA1c (HbA1c). Both the FPG and the 2Hr-PG are part of the Oral Glucose Tolerance Test (OGTT), but the FPG can be tested separately from the OGTT. Each test has several strengths and limitations. The FPG test is easy to perform in a clinic but relies on fasting overnight prior to blood draw. The 2Hr-PG measurements from the OGTT are more sensitive than FPG alone and capture post-meal glucose spikes indicative of diabetes. A blood sugar level that is less than 140 mg/dL is considered “normal,” whereas a reading of more than 200 mg/dL indicates diabetes. A reading between 140 and 199 mg/dL indicates prediabetes. However, the test relies on fasting and meal-prep, is time-consuming with at least two blood draws over 2 hours, and has relatively poor reproducibility (e.g., in the 60-80% range). Accordingly, repeating the OGTT is recommended to confirm diagnosis unless both the FPG and 2Hr-PG values are above diagnostic thresholds.

Although the RPG test can be performed at any time, it is not an accepted diagnostic on its own unless accompanied by overt diabetes symptoms. The HbA1c test does not rely on fasting and is accurate and reproducible. However, HbA1c is not a direct measure of glucose, and HbA1c values may be impacted by factors including anemias, hemoglobinopathies, race, and the so-called “glycation gap” corresponding to a disagreement between HbA1c and fructosamine measurements. These conditions, along with the suboptimal agreement between HbA1c and OGTT results, may lead to a misdiagnosis and delayed treatment, which may have adverse health and economic consequences.

Continuous glucose monitoring (CGM) systems report glucose levels from the blood or interstitial fluid at, for example, pre-determined intervals. As a non-limiting example, the pre-determined intervals are 5-minute intervals. In various implementations, the CGM system includes the analyte monitoring device 104. As such, additional details regarding the CGM system are described above with respect to FIGS. 1 and 2. An individual sensor session where the CGM system is worn continuously (e.g., without removal) may last for a pre-defined observation period, such as 10 days, and glucose concentration data can be made available to the CGM system wearer (e.g., user) to assist in diabetes management. Alternatively or additionally, sensor data can be recorded, but not displayed, for sessions run in “blinded mode.” Real-time CGM data include, for example, current glucose concentration estimates and trends that can inform immediate treatment decisions. Furthermore, retrospective analysis of the CGM data can yield summary statistics that inform longer-term interventions, adjustments, or risk of diabetes-related complications.

Given the limitations of current standard of care diagnostic tests and in accordance with the described techniques, an algorithm uses CGM data from an observation period as an alternative diagnostic for T2D. In the present example, a binary classification diagnostic CGM (dCGM) algorithm is used to generate classifications for T2D. In one or more implementations, HbA1c measurements are paired with tags classifying the measurements as corresponding to a diabetes status of normoglycemic, prediabetes, or T2D. Observed CGM-derived metrics may also be paired with observed HbA1c measurements so that a diabetes status can be paired in training data with such CGM-derived metrics. A wide range of CGM-derived metrics (e.g., features), including metrics for glycemic variability, time in-range, risk assessment scores, and so forth, may be paired with diabetes statuses for training the dGCM algorithm, in variations. Further examples of such metrics are represented by the extracted analyte features 416 and described with respect to FIG. 4. In one or more implementations, feature importance is evaluated in k-fold cross-validation, and desired CGM metrics may be down-selected based on discriminative power, interpretability, and collinearity.

In one or more implementations, the dCGM algorithm is trained to utilize, as inputs, a combination of metrics that assess glycemic level (e.g., time above 140 mg/dL) and quantified variability (e.g., interquartile range). To further illustrate the utility of these CGM data-derived metrics, a correlation between the glycemic level and variability with respect to diabetes status is shown in FIG. 14. FIG. 14 depicts a plot 1400 of the percent time above 140 mg/dL (vertical axis) and the interquartile range (horizontal axis). A key 1402 denotes the diabetes status associated with each datapoint according to fill and shape. In this example, datapoints from normoglycemic users 1404 (e.g., users having HbA1c measurements less than 5.7%) are represented by black-filled inverted triangles, datapoints from prediabetes users 1406 (e.g., users having HbA1c measurements greater than or equal to 5.7% and less than 6.5%) are represented by diagonally shaded circles, and datapoints from T2D users 1408 (e.g., users having HbA1c measurements of at least 6.5%) are represented by white-filled triangles (e.g., no shading).

As demonstrated by the plot 1400, plotting the percent time above 140 mg/dL against the interquartile range results in separation of users with and without T2D. For example, the T2D users 1408 are relatively clustered at higher percent time above 140 mg/dL values and higher interquartile range values (e.g., in the top right of the plot 1400), while the normoglycemic users 1404 and the prediabetes users 1406 are relatively clustered at lower percent time above 140 mg/dL values and lower interquartile range values (e.g., in the bottom left of the plot 1400). Because time spent above 140 mg/dL is uncommon in healthy individuals, this CGM metric may be used as a threshold for diagnosing T2D or assessing the risk of developing T2D.

The wealth of information generated by CGM data measured under real-life conditions enables feature extraction and feature selection for training machine learning-based algorithms for screening diabetes and predicting diabetes risk, such as described in detail above with respect to FIGS. 4-7 and 11-13. Although the present example employs a factory-calibrated CGM system worn for several days and blinded, retrospective data from a single sensor wear session, other examples may use data from CGM sensors having other calibrations, other wear durations, more than one sensor wear session, and so forth. Further, although the present example describes a binary classification algorithm to diagnose T2D, the dCGM algorithm may be adapted to classify a type of diabetes, a risk of developing diabetes, a prediction of developing adverse health effects related to diabetes, and the like.

The percent time above 140 mg/dL and the interquartile range represent one example of an interpretable set of CGM metrics that have high discriminative power for the classification of those with and without T2D. However, it is to be appreciated that other combinations of different and/or additional metrics based on high-density CGM data may be used to resolve the differences in dysglycemia among these users in other dCGM algorithms.

Having described examples of procedures in accordance with one or more implementations, consider now an example of a system and device that can be utilized to implement the various techniques described herein.

Example of a System and Device

FIG. 15 illustrates an example of a system generally at 1500 that includes an example of a computing device 1502 that is representative of one or more computing systems and/or devices that may implement the various techniques described herein. This is illustrated through inclusion of the prediction system 114 at the platform level as well as at the individual computing device level. The prediction system 114 may be implemented at one level or the other or at least partially at both levels. The computing device 1502 may be, for example, a server of a service provider, a device associated with a client (e.g., a client device), an on-chip system, and/or any other suitable computing device or computing system.

The example of the computing device 1502 as illustrated includes a processing system 1504, one or more computer-readable media 1506, and one or more input/output (I/O) interfaces 1508 that are communicatively coupled, one to another. Although not shown, the computing device 1502 may further include a system bus or other data and command transfer system that couples the various components, one to another. A system bus can include any one or combination of different bus structures, such as a memory bus or memory controller, a peripheral bus, a universal serial bus, and/or a processor or local bus that utilizes any of a variety of bus architectures. A variety of other examples are also contemplated, such as control and data lines.

The processing system 1504 is representative of functionality to perform one or more operations using hardware. Accordingly, the processing system 1504 is illustrated as including hardware elements 1510 that may be configured as processors, functional blocks, and so forth. This may include implementation in hardware as an application specific integrated circuit or other logic device formed using one or more semiconductors. The hardware elements 1510 are not limited by the materials from which they are formed or the processing mechanisms employed therein. For example, processors may be comprised of semiconductor(s) and/or transistors (e.g., electronic integrated circuits (ICs)). In such a context, processor-executable instructions may be electronically-executable instructions.

The computer-readable media 1506 is illustrated as including memory/storage 1512. The memory/storage 1512 represents memory/storage capacity associated with one or more computer-readable media. The memory/storage 1512 may include volatile media (such as random access memory (RAM)) and/or nonvolatile media (such as read only memory (ROM), Flash memory, optical disks, magnetic disks, and so forth). The memory/storage 1512 may include fixed media (e.g., RAM, ROM, a fixed hard drive, and so on) as well as removable media (e.g., Flash memory, a removable hard drive, an optical disc, and so forth). The computer-readable media 1506 may be configured in a variety of other ways as further described below.

The input/output interface(s) 1508 are representative of functionality to allow a user to enter commands and information to the computing device 1502 and also allow information to be presented to the user and/or other components or devices using various input/output devices. Examples of input devices include a keyboard, a cursor control device (e.g., a mouse), a microphone, a scanner, touch functionality (e.g., capacitive or other sensors that are configured to detect physical touch), a camera (e.g., which may employ visible or non-visible wavelengths such as infrared frequencies to recognize movement as gestures that do not involve touch), and so forth. Examples of output devices include a display device (e.g., a monitor or projector), speakers, a printer, a network card, tactile-response device, and so forth. Thus, the computing device 1502 may be configured in a variety of ways as further described below to support user interaction.

Various techniques may be described herein in the general context of software, hardware elements, or program modules. Generally, such modules include routines, programs, objects, elements, components, data structures, and so forth that perform particular tasks or implement particular abstract data types. The terms “module,” “functionality,” and “component” as used herein generally represent software, firmware, hardware, or a combination thereof. The features of the techniques described herein are platform-independent, meaning that the techniques may be implemented on a variety of commercial computing platforms having a variety of processors.

An implementation of the described modules and techniques may be stored on or transmitted across some form of computer-readable media, such as the computer-readable media 1506. The computer-readable media may include a variety of media that may be accessed by the computing device 1502. By way of example, and not limitation, computer-readable media may include “computer-readable storage media” and “computer-readable signal media.”

“Computer-readable storage media” may refer to media and/or devices that enable persistent and/or non-transitory storage of information in contrast to mere signal transmission, carrier waves, or signals per se. Thus, computer-readable storage media refers to non-signal bearing media. The computer-readable storage media includes hardware such as volatile and non-volatile, removable and non-removable media and/or storage devices implemented in a method or technology suitable for storage of information such as computer readable instructions, data structures, program modules, logic elements/circuits, or other data. Examples of computer-readable storage media may include, but are not limited to, RAM, ROM, EEPROM, flash memory or other memory technology, CD-ROM, digital versatile disks (DVD) or other optical storage, hard disks, magnetic cassettes, magnetic tape, magnetic disk storage or other magnetic storage devices, or other storage device, tangible media, or article of manufacture suitable to store the desired information thereon and which may be accessed by a computer.

“Computer-readable signal media” may refer to a signal-bearing medium that is configured to transmit instructions to the hardware of the computing device 1502, such as via a network. Signal media typically may embody computer readable instructions, data structures, program modules, or other data in a modulated data signal, such as carrier waves, data signals, or other transport mechanism. Signal media also include any information delivery media. The term “modulated data signal” means a signal that has one or more of its characteristics set or changed in such a manner as to encode information in the signal. By way of example, and not limitation, communication media include wired media such as a wired network or direct-wired connection, and wireless media such as acoustic, RF, infrared, and other wireless media.

As previously described, the hardware elements 1510 and the computer-readable media 1506 are representative of modules, programmable device logic and/or fixed device logic implemented in a hardware form that may be employed in some embodiments to implement at least some aspects of the techniques described herein, such as to perform one or more instructions. Hardware may include components of an integrated circuit or on-chip system, an application-specific integrated circuit (ASIC), a field-programmable gate array (FPGA), a complex programmable logic device (CPLD), and other implementations in silicon or other hardware. In this context, hardware may operate as a processing device that performs program tasks defined by instructions and/or logic embodied by the hardware as well as a hardware utilized to store instructions for execution, e.g., the computer-readable storage media described previously.

Combinations of the foregoing may also be employed to implement various techniques described herein. Accordingly, software, hardware, or executable modules may be implemented as one or more instructions and/or logic embodied on some form of computer-readable storage media and/or by one or more hardware elements 1510. The computing device 1502 may be configured to implement particular instructions and/or functions corresponding to the software and/or hardware modules. Accordingly, implementation of a module that is executable by the computing device 1502 as software may be achieved at least partially in hardware, e.g., through use of the computer-readable media 1506 and/or the hardware elements 1510 of the processing system 1504. The instructions and/or functions may be executable/operable by one or more articles of manufacture (for example, one or more computing devices 1502 and/or processing systems 1504) to implement techniques, modules, and examples described herein.

The techniques described herein may be supported by various configurations of the computing device 1502 and are not limited to the specific examples of the techniques described herein. This functionality may also be implemented all or in part through use of a distributed system, such as over a “cloud” 1514 via a platform 1516 as described below.

The cloud 1514 includes and/or is representative of the platform 1516 for resources 1518. The platform 1516 abstracts underlying functionality of hardware (e.g., servers) and software resources of the cloud 1514. The resources 1518 may include applications and/or data that can be utilized while computer processing is executed on servers that are remote from the computing device 1502. The resources 1518 can also include services provided over the Internet and/or through a subscriber network, such as a cellular or Wi-Fi network.

The platform 1516 may abstract resources and functions to connect the computing device 1502 with other computing devices. The platform 1516 may also serve to abstract scaling of resources to provide a corresponding level of scale to encountered demand for the resources 1518 that are implemented via the platform 1516. Accordingly, in an interconnected device embodiment, implementation of functionality described herein may be distributed throughout the system 1500. For example, the functionality may be implemented in part on the computing device 1502 as well as via the platform 1516 that abstracts the functionality of the cloud 1514.

In some aspects, the techniques described herein relate to a method, wherein selecting the combination of features of the plurality of features based on the robustness metric and the performance metric further includes: filtering the plurality of candidate combinations of the plurality of features based on the performance metric of each of the plurality of candidate combinations relative to a performance threshold; and selecting a filtered candidate combination having a highest value for the robustness metric as the combination of features.

In some aspects, the techniques described herein relate to a method, wherein selecting the combination of features of the plurality of features based on the robustness metric and the performance metric further includes: selecting a candidate combination from the plurality of candidate combinations of features that maximizes a combination of the robustness metric and the performance metric as the combination of features.

In some aspects, the techniques described herein relate to a method, further including training the one or more machine learning models to predict the health condition classification using the outcome data of the user population.

In some aspects, the techniques described herein relate to a method, wherein the individual performance metric is higher for first feature than the second feature, and the individual robustness metric is higher for the second feature than the first feature.

In some aspects, the techniques described herein relate to a method, wherein the individual performance metric and the individual robustness metric are both higher for the first feature than the second feature.

In some aspects, the techniques described herein relate to a method, wherein at least one of the first feature and the second feature is a value-based feature of the analyte measurements.

In some aspects, the techniques described herein relate to a method, wherein the health condition classification is an indication describing a presence of a health condition, an absence of the health condition, a degree of severity of the health condition, adverse effects associated with the health condition, and/or clinical diagnostic values associated with the health condition.

In some aspects, the techniques described herein relate to a method, further including: generating a notification based on the health condition classification of the user.

In some aspects, the techniques described herein relate to a method, wherein the notification includes a report directed to a health care provider of the user that includes the health condition classification of the user.

In some aspects, the techniques described herein relate to a method, wherein the notification is output to the user and includes instructions to contact a health care provider of the user.

In some aspects, the techniques described herein relate to a device, wherein the analyte data of the user is measured by the analyte sensor over an observation period, and wherein the observation period spans a plurality of days.

In some aspects, the techniques described herein relate to a device, wherein the state of the user with respect to the health condition is at least one of an absence of the health condition, a presence of the health condition, a severity of the health condition, a type of the health condition, and a probability of the user experiencing an adverse effect associated with the health condition.

In some aspects, the techniques described herein relate to a method, including: obtaining analyte measurements of a user during an observation period via an analyte sensor of an analyte monitoring device worn by the user during the observation period; extracting a first feature and a second feature from the analyte measurements of the user; providing a combination of the first feature and the second feature to a machine learning model; and receiving a prediction of a health condition classification from the machine learning model.

In some aspects, the techniques described herein relate to a method, further including generating a notification based on the prediction of the health condition classification received from the machine learning model.

In some aspects, the techniques described herein relate to a method, wherein generating the notification includes generating at least one of a first notification that is output to the user and includes instructions that do not include the prediction of the health condition classification and a second notification that is output to a health care provider of the user and includes the prediction of the health condition classification.

In some aspects, the techniques described herein relate to a method, wherein the first feature includes an amplitude-based feature, a time within range-based feature, a time outside of range-based feature, a stability-based feature, or an event occurrence-based feature.

In some aspects, the techniques described herein relate to a method, wherein the second feature includes a trend-based feature, a variability-based feature, a frequency-related feature, or an autocorrelation feature.

In some aspects, the techniques described herein relate to a method, wherein the health condition classification is a diabetes classification, and the analyte measurements are glucose measurements.

In some aspects, the techniques described herein relate to a method, wherein the diabetes classification is an indication describing a state of the user during the observation period as having gestational diabetes or no gestational diabetes.

In some aspects, the techniques described herein relate to a method, wherein the diabetes classification is an indication describing a state of the user during the observation period as having diabetes, no diabetes, or prediabetes.

In some aspects, the techniques described herein relate to a method, wherein the diabetes classification is an indication of one or more adverse effects of diabetes the user is predicted to experience.

In some aspects, the techniques described herein relate to a method, wherein the first feature is a value-based feature of the analyte measurements, and the second feature is a pattern-based feature of the analyte measurements.

In some aspects, the techniques described herein relate to a method, wherein each of the first feature and the second feature is a value-based feature of the analyte measurements.

In some aspects, the techniques described herein relate to a method, wherein each of the first feature and the second feature is a pattern-based feature of the analyte measurements.

In some aspects, the techniques described herein relate to a method, wherein the combination of the first feature and the second feature is selected based on a performance of the combination of the first feature and the second feature at predicting clinically determined health condition classifications of a user population while simulating manufacturing-related analyte sensor variance in historical analyte measurements of the user population.

In some aspects, the techniques described herein relate to a method, including: obtaining analyte measurements of a user during an observation period via an analyte sensor of an analyte monitoring device worn by the user during the observation period; extracting a feature from the analyte measurements of the user; providing the feature to a machine learning model; and receiving a prediction of a health condition classification from the machine learning model.

In some aspects, the techniques described herein relate to a method, further including selecting the feature based on a robustness metric associated with an insensitivity to manufacturing variabilities of the analyte sensor and a performance metric associated with predicting the health condition classification.

In some aspects, the techniques described herein relate to a method, further including generating at least one of a first notification including instructions that do not include the prediction of the health condition classification and a second notification that includes the prediction of the health condition classification.

Conclusion

Although the systems and techniques have been described in language specific to structural features and/or methodological acts, it is to be understood that the systems and techniques defined in the appended claims are not necessarily limited to the specific features or acts described. Rather, the specific features and acts are disclosed as examples of forms of implementing the claimed subject matter.

What is claimed is:

1. A method, comprising:
obtaining a plurality of features of analyte measurements;
selecting a combination of features of the plurality of features based on a robustness metric associated with an insensitivity to manufacturing variabilities of analyte sensors and a performance metric associated with predicting a health condition classification; and
training one or more machine learning models to predict the health condition classification using the combination of features.

2. The method of claim 1, wherein the analyte measurements are historical analyte measurements from a user population associated with outcome data of the user population, and wherein selecting the combination of features of the plurality of features based on the robustness metric and the performance metric comprises:
generating a model prediction of the health condition classification for each of a plurality of candidate combinations of the plurality of features; and
determining the performance metric for each of the plurality of candidate combinations based on the model prediction of the health condition classification relative to the outcome data of the user population.

3. The method of claim 2, wherein the performance metric indicates one or both of a sensitivity for predicting the health condition classification and a specificity for predicting the health condition classification based on the model prediction of the health condition classification relative to the outcome data for each of the plurality of candidate combinations.

4. The method of claim 2, wherein the outcome data indicates a clinically determined health condition classification of each user of the user population.

5. The method of claim 2, wherein selecting the combination of features of the plurality of features based on the robustness metric and the performance metric further comprises:
simulating the manufacturing variabilities of the analyte sensors in the analyte measurements over a plurality of simulation rounds that each introduce a different percentage of simulated variability to the analyte measurements; and
determining the robustness metric for each of the plurality of candidate combinations based on a change in the performance metric per percentage of the simulated variability.

6. The method of claim 5, wherein simulating the manufacturing variabilities of the analyte sensors in the analyte measurements over the plurality of simulation rounds comprises simulating different performance variabilities and analyte sensor characteristics to introduce the different percentage of simulated variability to the analyte measurements each simulation round.

7. The method of claim 2, wherein selecting the combination of features of the plurality of features based on the robustness metric and the performance metric further comprises:
filtering the plurality of candidate combinations of the plurality of features based on the robustness metric of each of the plurality of candidate combinations relative to a robustness threshold; and
selecting a filtered candidate combination having a highest value for the performance metric as the combination of features.

8. The method of claim 1, wherein the combination of features of the plurality of features comprises a first feature and a second feature, and wherein the method further comprises determining an individual performance metric and an individual robustness metric for each of the plurality of features using model predictions of the health condition classification for each of the plurality of features.

9. The method of claim 8, wherein at least one of the first feature and the second feature is a trend-related feature of the analyte measurements.

10. The method of claim 8, wherein the second feature is a variability and stability feature of the analyte measurements.

11. The method of claim 1, further comprising:
after training the one or more machine learning models to predict the health condition classification using the combination of features:
obtaining new analyte measurements from an analyte measurement device worn by a user over an observation period;
extracting features of the combination of features from the new analyte measurements;
inputting the extracted features of the combination of features into the one or more machine learning models; and
receiving, as an output of the one or more machine learning models, the health condition classification of the user.

12. A device comprising:
one or more processors; and
a memory having stored thereon computer-readable instructions that are executable by the one or more processors to perform operations comprising:
obtaining analyte data of a user that is measured by an analyte sensor;
extracting at least two features of the analyte data, the at least two features included in a multivariate model of analyte features determined to be robust to manufacturing variabilities of the analyte sensor based on variance simulations performed on historical analyte data of a user population;
inputting a combination of the at least two features to a machine learning model;
predicting, via the machine learning model, a health condition classification of the user; and
receiving, as an output of the machine learning model, the health condition classification.

13. The device of claim 12, wherein the analyte data of the user is measured by the analyte sensor over an observation period, and wherein the health condition classification is an indication describing a status of the user during the observation period with respect to a health condition.

14. The device of claim 13, wherein the health condition is diabetes, and wherein the health condition classification is one of a diabetes status, a prediabetes status, and a no diabetes status.

15. The device of claim 12, wherein the machine learning model is trained with training input portions comprising the combination of the at least two features extracted from the historical analyte data of the user population and expected output portions comprising labels representative of the health condition classification of each user of the user population.

16. A system comprising:
a wearable analyte monitoring device comprising a sensor that is inserted subcutaneously into skin of a user to collect analyte measurements of the user during an observation period;
a storage device to maintain the analyte measurements of the user collected during the observation period; and
a prediction system to predict a health condition classification of the user by extracting a robust analyte feature combination from the analyte measurements of the user and processing the robust analyte feature combination using one or more machine learning models.

17. The system of claim 16, wherein the one or more machine learning models are generated based on historical analyte measurements and historical outcome data of a user population, and the system further comprises a model manager to:
obtain the historical analyte measurements and the historical outcome data of the user population, the historical analyte measurements provided by analyte monitoring devices worn by users of the user population;
extract the robust analyte feature combination from the historical analyte measurements; and
generate the one or more machine learning models by:
providing the robust analyte feature combination extracted from the historical analyte measurements to the one or more machine learning models; and
adjusting weights of the one or more machine learning models based on a comparison of training health condition classifications received from the one or more machine learning models and clinically verified health condition classifications indicated by the historical outcome data.

18. The system of claim 17, wherein the clinically verified health condition classifications indicated by the historical outcome data are associated with one or more diagnostic measures independent of the historical analyte measurements provided by the analyte monitoring devices worn by users of the user population.

19. The system of claim 16, wherein one or both of the storage device and the prediction system is implemented, at least in part, at the wearable analyte monitoring device.

20. The system of claim 16, wherein the prediction system is implemented at one or more computing devices remote from the wearable analyte monitoring device.
